(19) United States
(12) Patent Application Publication
(10) Pub. No.: US 2023/0345992 A1
(43) Pub. Date: Nov. 2, 2023

(54) ORAL COMPOSITIONS OF LIPOPHILIC DIETY SUPPLEMENTS, NUTRACEUTICALS AND BENEFICIAL EDIBLE OILS

(71) Applicant: KARNAK TECHNOLOGIES, LLC, New York, NY (US)

(72) Inventor: Rafael Ezra, Tel Aviv (IL)

(21) Appl. No.: 17/779,036

(22) PCT Filed: Jul. 29, 2021
(86) PCT No.: PCT/IL2021/050914

§ 371 Date: May 23, 2022

Related U.S. Application Data
(60) Provisional application No. 63/058,278, filed on Jul. 29, 2020.

(51) Int. Cl. A23L 33/00; A23L 33/115; A23L 27/20; A23L 29/10; A23L 29/30

(52) U.S. Cl. CPC A23L 33/30; A23L 33/115; A23L 27/20; A23L 29/10; A23L 29/30

(57) ABSTRACT

The invention provides compositions that increase oral bioavailability of edible lipophilic substances such as beneficial edible oils, oil-soluble vitamins, and nutraceuticals. The compositions and methods of the invention are highly applicable to food industries in the production of foods, beverages, supplements, and food additives.

### TECHNOLOGICAL FIELD

The invention generally relates to compositions that increase oral bioavailability of edible lipophilic substances such as beneficial edible oils, oil-soluble vitamins, and nutraceuticals. The compositions and methods of the invention are highly applicable to food industries in the production of foods, beverages, supplements, and food additives.

### BACKGROUND

Many food and beverage industries use encapsulation technologies to improve water-dispersibility, chemical stability, and handling of hydrophobic ingredients, such as colors, flavors, lipids, nutraceuticals, preservatives, and vitamins. Particular interest incite lipophilic bio-actives such as vitamin A, D and E, β-carotene, lycopene, lutein, curcumin, resveratrol, and coenzyme Q10, wherein encapsulation is meant to provide improved oral bioavailability. But while emulsion-based technologies are relatively common in food industry, their application to edible delivery systems stills suffers from many drawbacks.

Specific problem with many hydrophobic bioactive compounds, including those found in natural food products, is their relatively low solubility, instability, and poor absorption in the gut, all materializing into low oral bioavailability. The problem of solubility is often resolved by the use surfactants. Traditionally, small molecule surfactants have been used in the food industry to enhance the formation and stability of emulsions. Recently, a number of additional applications have been identified based on the ability of surfactants to form micelles. In contrast to emulsions, micelles are thermodynamically stable systems. Yet, many studies suggest that micellar structures are not necessarily preserved in the acidic pH of the stomach. And more recent studies suggest that for certain lipophilic actives, surfactants may have counter effects regarding solubility versus intestinal membrane permeability.

Another popular approach to assist solubility of lipophilic actives is the use of cyclodextrins. Cyclodextrin-based formulations have gained widespread attention in the pharmaceutical industries. However, a more critical look suggests that cyclodextrins are not entirely predictable, and for some actives, they can lead to reduced absorption.

Overall, for many solubility enhancers, there is a tradeoff between their tendency to improve solubility of lipophilic actives and their propensity to have negative effects on the respective intestinal membrane permeability of the same actives. In other words, a successful delivery method is conditioned on careful choice of solubility enhancer(s) and combinations of other excipients, and their cumulative impact on physicochemical and biological properties of the resulting formulations.

Therefore, there is a clear incentive for the development of new and more progressive formulations of lipophilic substances for overcoming the drawbacks of solubility/permeability tradeoff. Even more challenging would be to propose a general and more inclusive approach for improving bioavailability of various types of lipophilic substances and actives, which would be more applicable to the food industry.

There are numerous publications describing certain types of oral formulations with various lipophilic actives in the academic and patent literature, including those applying nanotechnology. It seems however that none of them is sufficiently inclusive and adaptable so as to be applicable to a wide range of nutritionally relevant actives and to the processes of food manufacturing.

A specific problem is producing fine-crystal sugar. Formation of crystalline sugars plays an important role in many food products. Apart from the sensation of sweetness, sugars are also responsible for desirable textural properties of various foods. The art of controlling crystallization of sugars is one of the key elements in the production of successful sweets and other sugar containing food products.

Some food sugar products rely on the presence of a crystalline sugar, while in others the formation of sugar crystals is retarded. For example, the graining of hard candies is usually considered a defect and is usually avoided by specific formulations. On the other hand, ice creams and fondants require fine crystalline sugar for smoothness and creamy qualities, and to improve mixing.

Another example is chocolate. Chocolate is a suspension of fine particles in fat, consisting of cocoa solids, crystalline sucrose, and milk solids in milk chocolates. And while cocoa and milk solids are generally already fine enough, sucrose usually requires significant size reduction. The extra fine grade sucrose typically varies between 400 μm and 1000 μm. Therefore, as an ingredient in chocolate, the sucrose crystals must be reduced in size (<50 μm). Similar considerations apply to other types of confections.

The size reduction to micronic and sub-micronic range is a rapidly developing technology in the food industry. For solid particulate materials, micro- and nanonization usually involve various types of milling, grinding, and sieving. Liquid materials primarily use high-pressure and ultrasonic homogenization technologies. In general, reduction of particle size significantly enhances the physico-chemical and functional properties of food materials and leads to improvement of food quality.

With respect to sugar, grinding and sieving are energy intensive, expensive, and inefficient. When grinding and sieving crystalline sugar, the fracturing step usually yields a wide size distribution of crystals, which leads to re-grinding and sieving of large crystals and significant loses of the initial mass of sugar.

In-situ micronization is a novel particle engineering technique, whereby micron sized crystals are obtained during the process of production itself without the need for further particle size reduction. In contrast to other techniques requiring external processing conditions, like mechanical force, temperature, and pressure, with this technology the micronized product is obtained during the crystal formation.

Numerous publications related to methods of making sugar-based and sugar-coated food products. Nonetheless, it seems that none of them was instructive as to sufficiently straightforward and an accessible way to produce micronized sugar, while permitting certain degree of flexibility to include additional beneficial components imparting additional nutritional, flavoring and stability values to the end-product.

Certain types of oral formulations of lipophilic actives were previously described in WO20035850, WO2015/171445, WO2016/147186, WO2016/135621 and WO2017/180954 with actives such as cannabis, or isolated and pure cannabinoids. Examples of formulations using nanotechnology were described in WO19162951 and WO14176389 with solid formulations, in WO2013/108254 with liquid formulations, and in WO0245575 and WO03088894 with actives for uses in dentistry and cosmetics.

On sugar formulations in particular, WO20182789 described sugar-coated coacervate capsules with high content of disaccharides and encapsulated oil. WO11000827, US2010255154, JP2003339400 related to fortification of sugar with various bio-actives. None of them, however, provide a sufficiently straightforward and an accessible way to produce micronized sugar with additional beneficial components contributing nutritional, flavoring and stability values to the end product.

### GENERAL DESCRIPTION

The food market constantly demands new technologies to keep market leadership, and to produce fresh, authentic, convenient, and flavorful food products, with a prolonged shelf life, freshness, and quality. The new materials and products are anticipated to bring advancements and improvements to additional relevant sectors, impacting on agriculture and food production, food processing, distribution, storage.

Nanotechnology is an area of rising attention that unwraps new possibilities for the food industry. Nanotechnology is superior to the conventional food processing technologies as regards capabilities to produce foods with enhanced characteristics, quality, safety, and increased shelf life. Nanomaterials serve as a basis for qualitative and quantitative production of foods with enhanced bioavailability, tastes, textures, and consistencies, and new types of functional and medical foods.

With respect to poorly water-soluble or lipophilic substances, nano-delivery systems using specific solubility enhancers such as nanoemulsions, dendrimers, nano-micelles, solid lipid nanoparticles provide promising strategies for improving solubility, permeation, bio-accessibility, and oral bioavailability overall. Some of these systems further provide prolonged, and targeted delivery of actives.

Disadvantages of the conventional lipid-based formulations are well known, i.e., physical instability, limited active loading capacity, passive diffusion, active efflux in the gastrointestinal (GI) tract and extensive liver metabolism. Nanonization is one approach to solving these problems. The basic advantage of nanonization is in increasing the substrate surface area and dissolution rate. With lipophilic substances, nanonization can further increase saturation, solubility and reduce erratic absorption, thereby impacting on their transport through the GI wall and increasing their oral bioavailability.

Nanoencapsulation is a technology that packs substances into miniature structures using methods such nano-emulsification, and nano-structuration and production of nanocomposites to impart new qualities and/or new functionalities to the end-product. A specific example is nanoencapsulation of bio-actives and its applications in food industry. Encapsulation of food additives offers a range of abilities of making new tastes and controlling aroma release or masking unwanted tastes. It further enables to produce composite foods enriched in nutrients, supplements, and particularly those with poorly water-soluble actives such as lycopene, omega-3 fatty acids, β-carotene and isoflavones.

The present invention makes part of such emerging new technologies. The invention applies micro- and nanonization technologies to make and manipulate matter at a new size scale, and to create novel structures with highly unique properties and wide-ranging applications.

The primary goal of the invention has been to explore strategies for improving oral bioavailability of edible lipophilic substances, with tangible and provable applications in food industry. To that end, the invention provides an exclusive formulation approach which can be applicable to a wide range of lipophilic edibles and actives, such as edible oils, lipophilic vitamins, and natural extracts. The compositions of the invention, per se, can serve as a source of supplements and superfoods with higher loading of actives and improved oral bioavailability, and further as a basis for foods with higher nutritional value and novel desirable characteristics.

The oral compositions of the invention constitute a solid microparticulate matter which is fully dispersible in water. In other words, the microparticulate matter is generally not soluble in water, as described herein, and therefore can be formed into a water-based dispersion, as known in the art. This quality, per se, constitutes a significant advantage in terms stability, storage, operability, and applicability to food industry. Other properties of the compositions reside in the specific composition and arrangement of its core components, i.e., the sugars, the polysaccharides, the surfactants and the lipophilic nanospheres containing edible oils and/or other lipophilic actives. The present studies show that the oils and actives can be distributed inside and outside the lipophilic nanospheres, which is responsible for the feature of differential bioavailability characteristic of the compositions of the invention. The sugars, polysaccharides, and surfactants provide a formation or a porous mesh entrapping the lipophilic nanospheres. The formation or the porosity of the mesh can be modulated by the relative content of sugars, polysaccharides, surfactants, and oils, and the size of lipophilic nanospheres, which in turn impacts on the microparticulate structure and texture of the matter as a whole. Advantages of this particular structure have been revealed in surprising features of preservation of particles size upon dispersion in water, long-term stability, high loading capacity characteristic of the compositions of the invention.

Specific examples of the core components of the present compositions are trehalose, sucrose, mannitol, lactitol and lactose for sugars; maltodextrin and carboxymethyl cellulose (CMC) for polysaccharides; and ammonium glycyrrhizinate, pluronic F-127 and pluronic F-68 for surfactants. Regarding edible oils and actives, the compositions of the invention can use vegetable oils enriched in monounsaturated fatty acids (MUFAs) and polyunsaturated fatty acids (PUFAs), e.g., Omega-3 and Omega-6, and actives dissolved edible oils such as vitamins A, D, E and K, flavonoids, carotenoids, coenzyme Q10, probiotics, natural extracts and superfoods, and various combinations of such ingredients.

Thus, the compositions of the invention are essentially hybrid formulations combining the advantages of lipid-based formulations and nanoparticles in terms of high loading, long-term stability, reproducibility, enhanced bio-accessibility and oral bioavailability, and other properties.

All these structural and functional properties of the present compositions, as well as their applicability to various types of foods and food supplements have been presently explored and exemplified.

More specifically, the key feature of preservation of the original size of the nanospheres upon reconstitution of the powder compositions in water was found to be consistent throughout various processes of production, storage conditions and various composition of sugars, oils, and actives, and even upon fixation and release from water dissolvable films such as polyvinyl alcohol (PVA) (EXAMPLES 1-3).

First, the feature of reproducible nanometric size of the lipophilic nanospheres is highly surprising, especially in view of the known tendency of the nanoemulsion to increase particle size or fuse under various conditions. Second, it highly compatible with the food production processes which predominantly involve water. Third and the most important, it suggests that the benefits of nanonization can be preserved in the intestinal milieu, with the expected consequences of higher solubility, permeability, and bio-accessibility in situ (EXAMPLE 8).

Overall, it can be stated that the compositions of the invention provide consistent loading, entrapment, preservation and reconstitution capacities of oils and actives that are preserved through various exposures, manipulations, and conditions.

The feature of high loading capability was further addressed in a study showing that the compositions of the invention can be loaded with oils and actives up to 90%-95% of the total weight (w/w), this is without disrupting the core characteristics of preservation nanometric size in the reconstituted powder (EXAMPLE 5).

The feature of chemical preservation of actives was addressed in a study showing that the composition of the invention prevented degradation and oxidation of actives, even with actives sensitive to increased temperature, pro-oxidative species, and acidic pH such as lycopene and fish oil (EXAMPLE 4).

Further, another important feature of the compositions relates to different distributions of oils and actives inside and outside the lipophilic nanospheres and the ability to increase the encapsulation capacity (EXAMPLES 1.6-1.7) This feature is highly useful in providing compositions with differential bioavailability for the entrapped and the non-entrapped oils and actives. This feature was further supported by finding in vivo of bi-phasic release profiles of actives in plasma and tissues characteristic of the compositions of the invention (EXAMPLES 6-7).

A biphasic release pattern provides an immediate burst of active release and further a prolonged active release. Animals exposed to the compositions of the invention have consistently shown biphasic release profiles in plasma and tissues, while animals exposed to analogous lipid compositions showed only immediate release profiles. Due to limitations of the experimental time frame, the exact duration and nature (intermittent or sustained) of the prolonged release profiles remains to be established in future studies.

It can be stated that the immediate, prolonged, and potentially targeted release of actives are essential attributes of the present compositions, per se, as they arise from the specific composition and structure of their core components. Overall, these features are reflected in improved oral bioavailability of the present compositions over lipid forms with the same actives.

The concept of modulation of bioavailability is particularly applicable for vitamins, supplements, nutraceuticals, and superfoods, which are meant to achieve therapeutic objectives. Modified release formulations provide chosen characteristics of time course and/or location of active-release and have the potential to achieve desired therapeutic outcomes. Such products can further include carriers, excipients, and various types of coating to enhance consistency, viscosity, and taste to achieve better compliance.

Importantly, the compositions of the invention permit modulation the release profiles by changing the distribution of oils and actives inside and outside the lipophilic nanospheres, and modulation of encapsulation capacity. Encapsulation oils and actives is dependent on the amounts and types of oils and/or the amount and types of sugars, polysaccharides, and surfactants. It can be enhanced by removal of the non-encapsulated oil with hexane, for example.

In other words, the amount and/or the proportion of oil governs the structure of the composition and the distribution of oil inside and outside the nanospheres, and thereby governs the differential availability of oil and lipophilic active. Therefore, by varying the amount and the proportion of oil (and actives) it would be possible to modulate the loading and encapsulation capacity of the composition and its oral bioavailability.

More specifically, the compositions of the invention can be provided with various distributions of oils and actives inside or outside the lipophilic nanospheres as far as ratios of between about 1:0 to 9:1, respectively, and more practically as ratios of between about 4:1, 7:3, 3:2, 1:1, 3:7 or 1:4, respectively.

It has been further demonstrated that the present formulation approach is applicable to various types of edible oils, combinations of oils and lipophilic actives, as single actives and also complex extracts and superfoods in various consistencies and forms (EXAMPLES 1-9). In addition, the compositions of the invention preserved their core properties after being embedded and then released from a sublingual PVA patch (EXAMPLE 3).

Thus, the presently proposed formulation approach offers a substantial degree of flexibility and applicability to numerous types of edible oils and substances generally characterized as lipophilic, in other words, the entire range of lipophilic foods and substances regulated under GRAS (Generally Recognized as Safe) and DSHEA (Dietary Supplement Health and Education Act).

Overall, the powder forms of the invention have been related to properties of higher loading, higher encapsulation capacity, higher stability, modulated release and improved oral bioavailability and bio-accessibility of actives, which significantly exceeded those related to analogous lipid-based compositions; this, with a minimum concentration of surfactants. In addition, in contrast to lipid-based compositions where there is a limited play with excipients, the compositions of the invention permit application of a full range of excipients. All these make the compositions of the inventions a promising approach for improving the in vitro and in vivo properties of edible oils and poorly soluble actives, thus making them highly relevant for applications in food industry.

Another problem solved by the present invention relates to the issue of micronization of sugar. To that end, the invention provides a smooth finely granulated sugar powder, which in itself is a composite particulate material made of a sugar crystalline matrix with entrapped lipophilic nanopsheres or nanodroplets. This particular structure confers to the composite the desired characteristics of sugar (e.g., taste, small crystals, larger surface area, higher solubility, mechanic and thermodynamic stability during processing and storage) and the ability to capture or entrap a variety of desired lipophilic actives to impart new qualities and functions to the end-product.

Encapsulation, apart from new flavors, aromas, colors and actives with enhanced nutritional value, can further impact on chemical or biological degradation of actives and prolongs shelf life. Another function is the potential of controlled and targeted delivery of specific actives. All these make nanoencapsulation an ideal technology for producing ‘functional foods’.

Micronization of sugar, per se, has many advantages. As has been noted, a wide number of food products use sugars for organoleptic and textural characteristics. The crystalline phase of sugar has significantly different textural properties, in addition to inadequate dispersion in any coloring dyes used in foods. Controlling the formation of sugar crystals, predominantly towards a minimization, is important in the process of manufacturing sweet products as well as in the design of new products.

Crystallization of sugar is a complex process. Conventional wisdom guides to crystallization of sugar by supersaturation. But implementation of supersaturation in a manufacturing process is heat and energy intensive. Moreover, nucleation of sugar crystals during supersaturation is almost uncontrollable, and usually results in crystals of various sizes and shapes.

As has been noted, some foods such as ice creams, chocolates and fondants, where sugar is suspended rather solubilized, require reduced-size crystalline sugars. The chocolates in particular use sugar crystals that smaller than 50 μm. The conventional methods of achieving this type of product are expensive and inefficient. The present invention, instead, offers a straightforward and practical method for producing a relatively uniform population of micronized sugar crystals with sizes within a micronic range, i.e., between about 10 μm and 200 μm, and even 20 and 50 μm.

To that end, the invention employs a micronization in situ approach, whereby the microcrystals are produced during the process of production itself, without additional steps of particle size reduction and ensuing losses of energy and material. There is relatively little experience with the application of micronization in situ in food industry. Applicability of this technology for producing food products with improved properties of size, texture, dissolution, and taste has been presently exemplified (EXAMPLE 10).

Another important property is versatility or the ability to control particles size. Owing to their particular composite structure, there is a positive correlation between the size of the sugar particles and the size of the entrapped lipophilic nanospheres. Evidence for the existence of such correlation has been provided in the present examples (EXAMPLE 10.3). Therefore, the presently proposed method of making sugar is not only advantageous in terms of ability to provide a superior product but also in terms of ability to modify or adapt the product to specific applications and needs.

Thus, the technology invention offers a platform for making a range of sugar products with predetermined or carefully controlled particle size and oil content to provide improved qualities to the known food products, and further to design and develop completely new products with new and enhanced properties with a range of possibilities and future applications.

From yet another point of view, the invention provides an exclusive formulation approach to resolve known problems with formulating lipophilic edibles substances, actives, colors, flavors, nutraceuticals, stabilizers and vitamins. Poor water-dispersibility, stability and efficacy of lipophilic actives are well known. Nutraceuticals and vitamins in particular, e.g., vitamins A, D, E, β-carotene, lycopene, curcumin, resveratrol and coenzyme Q10, suffer from drawbacks of poor bio-solubility, chemical instability, poor absorption, and low oral bioavailability. Encapsulation and nanonization are potential approaches for improving bio-delivery of such actives.

The invention offers a composite approach: (1) encapsulation and nanonization to facilitate the bio-delivery of lipophilic actives, flavors, stabilizers, and (2) production of a micronized porous sugar material to incorporate these structures into edibles and attractive foods and other products. These two elements are mutually interactive in terms of size. The potential for incorporating various supplements and vitamins into the lipophilic nanospheres has been presently exemplified.

As has been noted, apart from sugars and oils, this structure is facilitated by several additional components, specifically the polysaccharides and surfactants. Specific characteristics of all these components will be discussed in detail below. It should be noted that the compositions can comprise various representatives of these groups, from different sources and in various combinations.

Further, the exact proportions of components can vary depending on the desired characteristics of taste, texture, nutritional value, and other qualities. The respective concentrations can be broadly characterized as: in the range of 30%-80% for the sugars, 10%-80% for the oils, 5%-25% for the polysaccharides and about 1%-10% for the surfactant, respectively, as per the dry weight of the composition (w/w).

Specific examples of compositions comprising sucrose, maltodextrin, sugar ester (SP30) and Theobroma oil (cocoa butter) within the specified concentrations ranges have been presently exemplified.

The compositions can further comprise a range of lipophilic substances encapsulated in the lipophilic nanospheres. Specific examples are lipophilic nutraceuticals, vitamins, dietary supplements, antioxidant, superfoods and extracts of animals or plants, probiotic microorganisms and in various proportions and combinations. Additional examples are lipophilic food colorants, taste and aroma enhancers, taste maskers, and food preservatives.

Nanoencapsulation further implies that the compositions can include carriers, excipients for preservation of specific properties, such as stability, shelf life, taste, etc., and other ingredients facilitating absorption and controlled release of actives.

In the broadest sense, the present technology provides a composite of a porous material containing entrapped nanoparticles, wherein the porous material and the nanoparticles are opposites in terms of hydrophobicity/hydrophilicity. In other words, the technology can provide a composite material made of a hydrophilic porous material with hydrophobic nanoparticles, and vice versa, a composite made of a hydrophobic porous material with hydrophilic nanoparticles. This versatility stems from the specific ingredients of the composite material, i.e., one or more types of sugars, oils, polysaccharides and surfactants.

From yet another point of view the present technology provides a ‘smart food’ or a ‘functional food’ using nanoencapsulation to entrap hydrophobic or hydrophilic materials and thereby impart specific desirable properties to the end food product. Furthermore, the technology uses an encapsulated core as means to control the size of the encapsulated nanoparticles, thereby conferring a desired granulation, solubility texture and taste and additional properties to the food product.

Ultimately, the invention builds on the concept of food on-demand. The idea of specifically tailored or interactive food can allow consumers to modify food depending on their own nutritional needs or tastes. For example, nowadays people are requiring more nutritional supplements in more specific and customized proportions, in view of the differences between the absorption in infants, children, adults, elderly and people suffering from gastrointestinal diseases.

The compositions and methods of the invention can make a difference not only in terms of better-quality food products as regards taste, texture, shelf life and ways of food processing, but also in terms of better safety and health benefits that such foods are bound to deliver. It offers a new platform for designing new and advanced food products with improved qualities and enhanced nutritional value, and innovative delivery systems for lipophilic edible products and other lipophilic actives.

### BRIEF DESCRIPTION OF THE DRAWINGS

To better understand the subject matter and to exemplify how it may be carried out in practice, embodiments will now be described by way of non-limiting examples with reference to the following drawings.

FIG. 1 illustrates the feature of preservation of particle size characteristic of the powder compositions of the invention. Figure shows powder compositions comprising cannabinoids (THC or CBD) stored at 45° C. (oven) for 1, 35, 54, 72 and 82 days (3 months correlates to 24 months at RT).

FIG. 2 illustrates the feature of protection of lipophilic actives and oils imparted by the present powder compositions. Figure shows TOTOX (overall oxidation state) values for fish oil (dashed) and the powder composition comprising the same (solid). Fish oil is sensitive to oxidation. Figure shows significantly lower levels of the primary and secondary oxidation products in the fish oil formulated into the powder composition starting from day 0 and up to day 14.

FIGS. 3A-3B illustrate the advantages of improved oral bioavailability of actives CBD (A) and THC (B) with the powder compositions (LL-P) compared to the lipid-based compositions with the same actives (LL-OIL), as revealed after single oral dose administration in a rat model. Figures show biphasic release profiles in plasma characteristic of the compositions of the invention, providing immediate and prolonged release and improved release of actives overall.

FIGS. 4A-4D show that the advantages of improved oral bioavailability are reproduced in tissues of animals administered with the powder compositions (LL-P) with THC and CBD and lipid-based compositions with the same actives (LL-OIL). Figures show the characteristic bi-phasing active release profile in the liver and brain.

FIG. 5 shows that the advantages of improved oral delivery and bioavailability are applicable to a wide range of lipophilic actives and oils. Figure shows actives release profile in plasma of the powder Vitamin D3 composition (solid) vs. the analogous lipid composition (dashed) upon single oral dose administration in a rat model. The powder composition shows a 2-fold increase in the concentration of Vitamin D3 over the lipid composition.

FIG. 6 illustrates the feature of enhanced bio-accessibility (degree of GI digestion) characteristic of the compositions of the invention using semi-dynamic in vitro digestion model. Figure show enhanced bio-accessibility of two actives found in Oregano, Thymol and Carvacrol, of the powder compositions (P) compared to the respective oil forms (O), for each active and total actives.

FIGS. 7A-7D further expand on the advantages of improved bio-accessibility using semi-dynamic model. Figures show that the protective effect and bio-accessibility of the powder composition can be further enhanced with enteric coated capsule (solid) compared the powder composition alone (dashed) and the oil-based composition (dotted). Figure relates to the bio-accessibility of total Thymol and Carvacrol (A), Carvacrol (B) and Thymol (C) at the end of the gastric phase, and the bio-accessibility of total Thymol and Carvacrol in the powder composition with enteric coated capsule (D) during the gastric and duodenal phases.

FIGS. 8A-8B are SEM images (scanning electron microscope) under magnification ×1K (A) and ×5K (B) showing sugar particles with Theobroma oil with the characteristic smooth, finely granulated texture, and size in the range of 20-50 μm.

FIGS. 9A-9D illustrate the composite nature of the sugar particle of the invention. Figures are cryo-TEM images (cryogenic transmission electron microscopy) showing lipophilic nanospheres of average size of 80-150 nm entrapped in the sugar particle.

FIGS. 10-11 illustrate the feature of controlling the sugar particle size by the size of entrapped lipophilic nanospheres. The size of the nanospheres can be modified within the range of about 50-900 nm by intensity of emulsification and pressure.

FIGS. 10A-10B are SEM images under magnifications ×1K (A) and ×0.5K (B) showing sugar particles with Theobroma oil produced under emulsification conditions wherein nanospheres had average size of 800 nm, yielding sugar particles with average size in the range of 130-160 μm.

FIGS. 11A-11B are SEM images under magnifications ×1K (A) and ×0.5K (B) wherein the entrapped nanospheres had average size of 150 nm and the resulting sugar particles had average size in the range of 20-50 μm.

FIG. 12 illustrates the feature of enhanced sweetness characteristic of the powder forms of the invention. Figure shows the results of organoleptic test of the Theobroma oil composition of the invention, whereby all 4 tasters reported 15% to 30% enhanced sweetness for the composition of the invention compared to sucrose.

FIG. 13 illustrates the feature of enhanced melting in the mouth characteristic of the powder forms of the invention, as revealed in the same organoleptic test. All 4 tasters reported an enhanced sensation of melting for the composition of the invention (light grey) compared to sucrose (dark grey).

FIG. 14 shows in vitro dissolution test comparing 4 types of powders: sucrose:maltodextrine 8:2 (w/w), finely crushed sucrose:maltodextrine 8:2 (w/w), micropowder of Theobroma oil and nanopowder of Theobroma oil, with the nanopowder of the invention showing the fastest dissolution rate.

### DETAILED DESCRIPTION OF EMBODIMENTS

It should be appreciated that the invention is not limited to specific methods, and experimental conditions described herein, and that the terminology used herein is for the purpose of describing particular embodiments only and is not intended to be limiting, since the scope of the present invention is determined only by the appended claims.

Many researchers and industries are currently developing various delivery systems to increase the oral bioavailability of lipophilic bioactive agents, such as oil-soluble vitamins, nutraceuticals, and lipids. Due to their poor solubility, there are significant challenges associated with incorporating these different bio-actives into foods, beverages, and other consumable forms. Different nanoemulsion fabrication methods have been employed for improving the stability and oral bioavailability of various kinds of hydrophobic vitamins and nutraceuticals.

One of the main disadvantages of nanoemulsions, in general, is their relative instability in terms of particles size over time. The nanoemulsions in solid powder forms, which are considered advantageous for oral administration, are renowned for this lack of uniformity in particle size, and particularly after reconstitution in water. Apart from the non-uniformity, there is a general tendency to increase particle size due to fusion or reconstruction of particles, thus reducing the overall surface area.

An increased particle size and lack of uniformity lead to significant variability in the absorption of substances entrapped in the nanoparticles, and poor oral bioavailability. The larger particles with smaller surface area have an inferior absorption in plasma and tissues. Therefore, despite the potential of the nanoemulsion technology, there are still significant drawbacks with its incorporation into the industry of foods, beverages, and other food products.

The present invention has proved to surpass these difficulties with nanonized powder compositions of edible oils and additional edible lipophilic actives, which while being readily dispersible in water preserve properties of loading, encapsulation and storage potential and improved oral bioavailability.

In the broadest sense, the compositions of the invention can be articulated as oral solid water-dispersible compositions of edible lipophilic substances, which can be edible oils and edible substances added or dissolved in such oils, such as lipophilic supplements, antioxidants, vitamins, nutrients, superfoods, and other additives.

In other words, in numerous embodiments the compositions of the invention can comprise an edible oil or a composition of edible oils.

In other embodiments the compositions of the invention can comprise one or more edible lipophilic substances or actives dissolved in edible oils.

In this context, substances that are applicable to the invention do not include conventional therapeutic products, strictly pharmaceutical products or actives, or human drugs regulated under FDA or EMA (the European equivalent).

The term ‘ [symbol] ’ relates to the feature of lipophilicity or the ability of a chemical compound to dissolve in fats, oils, lipids, and non-polar solvents. Lipophilicity, hydrophobicity, and non-polarity may describe the same tendency, although they are not synonymous. Lipophilicity of uncharged molecules can be estimated experimentally by measuring the partition coefficient (log P) in a water/oil biphasic system (e.g., water/octanol). For weak acids or bases, the measurements must further consider the pH at which most of the species remain unchanged vs. at which most of the species are charged. A positive value for log P denotes a higher concentration in the lipid phase (i.e., the compound is more lipophilic).

Thus, in numerous embodiments, the invention applies to uncharged or weekly charges lipophilic substances having a partition coefficient (log P) of more than 0.

More specifically, the invention is applicable to any edible lipophilic substance with a log P in the range between 0-1, 1-2, 2-3, 3-4, 4-5, 5-6, 6-7, 7-8, 8-9, 9-10, 10-11, 11-12, 12-13, 13-14, 14-15, 15-16, 16-17, 17-18, 18-19, 19-20, or more.

The term ‘ [symbol] ’ encompasses herein any dietary fats and oils, both from animal and plant sources, e.g., as triacylglycerols. In general, fats of animal origin tend to be relatively high in saturated fatty acids, contain cholesterol and are solids at room temperature. Oils of plant origin tend to be relatively high in unsaturated (mono- and polyunsaturated) fatty acids and are liquids at room temperature.

In numerous embodiments the compositions of the invention can comprise natural oils obtained from a vegetable or an animal source, or mixtures thereof.

Yet in other embodiments the compositions of the invention can comprise synthetic oils or fats, or mixtures thereof with the natural oils.

In numerous embodiments the compositions of the invention can comprise edible oil that are solid, semi-solid and/or liquid at room temperature.

Notable exceptions include plant oils, termed tropical oils (e.g., palm, palm kernel, coconut oils), and partially hydrogenated fats. Tropical oils are high in saturated fatty acids but remain liquid at room temperature because of high proportions of short-chain fatty acids. Partially hydrogenated plant oils are relatively high in trans fatty acids.

Edible oils further contain small amounts of antioxidants. Examples of natural antioxidants are tocopherols, phospholipids, ascorbic acid (vitamin C), phytic acid, phenolic acids and others. Common synthetic antioxidants for edible use are butylated hydroxyanisole (BHA), butylated hydroxytoluene (BHT), propyl gallate (PG), tertiary butyl hydroquinone (TBHQ), etc. The invention encompasses all these as well.

Dietary fats and oils differ in the chain lengths of their constituent fatty acids as saturated (SFAs), monounsaturated (MUFAs) and polyunsaturated (PUFAs) fatty acids. These differences markedly affect concentrations of lipids in plasma and the level of plasma cholesterol. When SFAs are replaced by unsaturated fats, total plasma cholesterol is lowered. As such, replacement of SFAs with polyunsaturated fatty acids and increased consumption of Omega-3 fatty acids from fish and plant sources have been associated with reduced risk for coronary heart disease.

The composition and type of fatty acids can be determined by gas-liquid chromatography (GLC), GLC combined with mass spectrometry. high-liquid chromatography (HPLC), for example.

In numerous embodiments, the oils that are applicable to the compositions of the invention are predominantly unsaturated oils, or oils comprising a substantial proportion of monounsaturated fatty acids (MUFAs) and polyunsaturated fatty acids (PUFAs).

In numerous embodiments, the edible oils are obtained from fish and plant sources, which are enriched in Omega-3 fatty acids. More specifically, there are three types of Omega-3 fatty acids: Eicosapentaenoic acid (EPA), Docosahexaenoic acid (DHA) and alpha-Linolenic acid (ALA). Thus, in numerous embodiments, the edible oils of the invention can naturally contain or be enriched with at least one of the Omega-3 fatty acids, or any combinations from that list.

In numerous embodiments, an oil of choice can be olive oil, which is appreciated for both taste and health properties, especially the extra-virgin category. The olive oil is rich in MUFAs, in Omega-3 and Omega-6 fatty acids.

Omega-3 and Omega 6 fatty acids play crucial role in brain function, normal growth and development. Omega-6 types help stimulate skin and hair growth, maintain bone health, regulate metabolism and reproductive system. Omegas 6 are present in safflower oil, sunflower oil, corn oil, soybean oil, sunflower and pumpkin seeds, walnuts.

A non-limiting list of edible oils that are applicable to the invention includes, among others, coconut oil, corn oil, canola oil, cottonseed oil, olive oil, palm oil, peanut oil, rapeseed oil, safflower oil, sesame oil, soybean oil, sunflower oil, almond oil, beechnut oil, brazil nut oil, cashew oil, hazelnut oil, macadamia oil, mongongo nut oil, pecan oil, pine nut oil, pistachio oil, walnut oil, pumpkin seed oil, grapefruit seed oil, lemon oil, orange oil, argan oil, avocado oil, and other well-known vegetable oils, and further non-vegetable oils from fish, such as herring oil, sardine oil, mackerel oil, salmon oil, tuna oil, halibut oil, swordfish oil, green shellfish oil, tilefish oil, pollock fish oil, codfish oil, catfish fish oil, snapper fish oil and flounder fish oil.

In numerous embodiments the compositions of the invention can comprise one or more edible oils selected from canola oil, sunflower oil, sesame oil, peanut oil, grapeseed oil, ghee, avocado oil, coconut oil, pumpkin seed oil, flaxseed oil, hemp oil, olive oil.

An extended list of edible oils that relevant to the present compositions is provided in ANNEX A.

From another point of view, the oral compositions of the invention can be seen as a composite matter comprising a plurality of micrometric particles each comprising a plurality of lipophilic nanospheres with an average size in the range of about 50 nm to about 900 nm and one or more edible lipophilic substances that are contained in the micrometric particles and are distributed inside and/or outside the lipophilic nanospheres at predetermined proportions, thereby providing immediate and/or prolonged delivery of the at least one edible lipophilic substance.

In other words, the compositions of the invention are a solid particulate matter comprising particles at a micrometric scale, or particles with an average size in a range of between about 10-900 μm, or more specifically with an average size in the range of 10-100 μm, 100-200 μm, 200-300 μm, 300-400 μm, 400-500 μm, 500-600 μm, 600-700 μm, 700-800 μm and 800-900 μm.

In certain embodiments the powders of the invention can comprise particles with an average size in a range of between about 10 μm and to about 300 μm, or more specifically with an average size in the range of 10-50 μm, 50-100 μm, 100-150 μm, 150-200 μm and 250-300 μm.

The micrometric particles of the compositions of the invention, in themselves, are a composite matter comprising lipophilic nanospheres with an average size between about 50-900 nm, and more specifically, an average size in a range between about 50-100 nm, 100-150 nm, 150-200 nm, 200-250 nm, 250-300 nm, 300-350 nm, 350-400 nm, 400-450 nm, 450-500 nm, 500-550 nm, 550-600 nm, 650-700 nm, 700-750 nm, 750-800 nm, 800-850 nm, 850-900 nm and 900-1000 nm (herein an average size is an average diameter).

The size or diameter of the lipophilic nanospheres can be measured by DLS (dynamic light scattering) upon reconstitution of the powder composition in water, such measurements have been presently exemplified.

In numerous embodiments the size of the micrometric particles correlates to the size of the lipophilic nanospheres, meaning that the size of the lipophilic nanospheres governs the size of the of the micrometric particles.

The above implies that the lipophilic nanospheres are essentially entrapped in the micrometric particles. It further implies that this composite matter has certain porosity or arrangement permitting to contain the nanospheres. These two features have been presently exemplified. They are further reflected in the loading and the encapsulation capacity characteristic of the compositions of the invention (see below)

An important feature of the invention is that the shape and size of the lipophilic nanospheres are substantially maintained upon dispersion in water. In other words, due to particular composition and structure of the composite matter, the average size of the nanospheres remains unchanged under various conditions such as lyophilization, long-term storage, fixation and release from matrixes or films such as PVA, etc. The term ‘ [symbol] ’ herein implies a deviation of 1-5%, 5-10%, 10-15%, 15-20% or up to 25% in average diameter before and after the manipulation or exposure to certain conditions.

An important feature of the present compositions resides in the distribution of the edible lipophilic substances inside and outside the lipophilic nanospheres. This feature is responsible for the properties of immediate and/or prolonged delivery or of release of actives characteristic of the compositions of the invention.

In numerous embodiments the edible lipophilic substances can be distributed inside or outside the lipophilic nanospheres at a ratio of between about 1:0 to 9:1, respectively.

In certain embodiments the edible lipophilic substances can be distributed inside or outside the lipophilic nanospheres at a ratio of between about 4:1, 7:3, 3:2, respectively, meaning that they are present in an excess inside the lipophilic nanospheres.

In other embodiments the edible lipophilic substances can be distributed inside or outside the lipophilic nanospheres at a ratio of between about 3:7 or 1:4, respectively, meaning that they are present in an excess outside the lipophilic nanospheres.

In still other embodiments the edible lipophilic substances can be distributed inside or outside the lipophilic nanospheres at the ratio of about 1:1, meaning that they are present in approximately equal proportions inside and outside the lipophilic nanospheres.

The same feature can be further articulated in terms of encapsulation capacity of the edible lipophilic substances into the compositions. The term ‘encapsulation capacity’ refers to the amount or a proportion of edible lipophilic substances that are entrapped inside the particulate matter, or the powder composition as a whole.

In numerous embodiments the compositions of the invention can have an encapsulation capacity of edible lipophilic substances up to at least about 80% (w/w) relative to the total weigh, or more specifically up to at least about 50%, 55%, 60%, 65%, 70%, 75%, 80%, 85%, 90%, 95%, 96%, 97% and 98% (w/w), or in the range between about 50%-98%, 60%-98%, 70-98%, 80-98% and 90-98% (w/w) relative to total weigh.

This feature can be further articulated as the encapsulation capacity of up to at least about 80% (w/w) relative to the weight of the oil component, or more specifically up to at least about 50%, 55%, 60%, 65%, 70%, 75%, 80%, 85%, 90%, 95%, 96%, 97% and 98% (w/w), or in the range between about 50%-98%, 60%-98%, 70-98%, 80-98% and 90-98% (w/w) relative to the weight of the oil component.

This feature is further related to loading capacity of the edible lipophilic substances onto the compositions. The term ‘ [symbol] ’ refers to the amount or a proportion of edible lipophilic substances that are loaded onto the powder composition.

In numerous embodiments the compositions of the invention can have a loading capacity of edible lipophilic substances up to at least about 80% (w/w) relative to total weigh, or more specifically up to at least about 50%, 55%, 60%, 65%, 70%, 75%, 80%, 85%, 90%, 95%, 96%, 97% and 98% (w/w), or in the range between about 50%-98%, 60%-98%, 70-98%, 80-98% and 90-98% (w/w) relative to total weigh.

This feature can be further articulated as the loading capacity of up to at least about 80% (w/w) relative to the weight of the oil component, or more specifically up to at least about 50%, 55%, 60%, 65%, 70%, 75%, 80%, 85%, 90%, 95%, 96%, 97% and 98% (w/w), or in the range between about 50%-98%, 60%-98%, 70-98%, 80-98% and 90-98% (w/w) relative to the weight of the oil component.

Another important feature characteristic of the present compositions is long-term stability or an extended shelf-life. This feature encompasses herein structural, chemical, and functional stabilities. In this instance, the structural stability is reflected in the ability to preserve particle size of the nanospheres upon reconstitution in water. The chemical stability reflects protection against degradation and oxidation under temperature, light and acidic pH, for example. The functional stability is reflected in preservation of properties of immediate and prolonged actives release.

In numerous embodiments the compositions of the invention can have a long-term stability of about at least about 1 year at room temperature, or more specifically up to at least about 6 months, 1 year, 2, years, 3 years, 4 years, 5 years at room temperature.

With respect to the other obligatory components of the present compositions. In numerous embodiments, apart from edible lipophilic substances, the compositions of the invention comprise at least one edible sugar, at least one edible polysaccharide and at least one edible surfactant. These other components are essentially responsible for the arrangement and porosity of the composite matter, and together with the oil component impact on the features of preservation of particle size, loading and encapsulation capacity characteristic of the present compositions.

In certain embodiments the edible sugar can be selected from trehalose, sucrose, mannitol, lactitol and lactose.

In certain embodiments the edible polysaccharides can be selected from maltodextrin and carboxymethyl cellulose (CMC).

In certain embodiments the edible surfactants can be selected from ammonium glycyrrhizinate, pluronic F-127 and pluronic F-68.

In numerous embodiments the compositions of the invention can comprise other types of edible sugars, polysaccharides, and surfactants.

For example, sugars that are applicable to present technology can be broadly characterized as short chain carbohydrates and sugar alcohols, and more specifically oligo-, di-, monosaccharides and polyols. Specific examples of such sugars, in addition to those mentioned above, are xylitol, sorbitol, maltitol.

The polysaccharides can include fructans found in many grains and galactans found in vegetables, and further polysaccharides as methyl-, carboxymethyl- and hydroxypropyl methyl-celluloses, and also pectin, starch, alginate, carrageenan, and xanthan gum.

The surfactants can include edible nonionic and anionic surfactants such as cellulose ether and derivatives, citric acid esters of mono- and diglycerides of fatty acids (CITREM), diacetyl tartaric acid ester of mono- and diglycerides. Additional examples of edible surfactants used in food industry are polysorbate 80 and lecithin.

In certain embodiments the compositions of the invention can comprise edible surfactants selected from monoglycerides, diglycerines, glycolipids, lecithins, fatty alcohols, fatty acids, or mixtures thereof.

In certain embodiments the compositions of the invention can comprise at least one edible surfactant which is a sucrose fatty acid ester (sugar ester).

It should be noted that the compositions of the invention can comprise any combination of the above components in various concentrations and proportions, with more than one candidate from the above groups.

An extended list of edible polysaccharides and surfactants that relevant to the present compositions is provided in ANNEX A.

More generally, in numerous embodiments the edible lipophilic substances can constitute between about 10% to about 98% of the compositions of the invention (w/w), or more specifically between about 10%-20%, 20%-30%, 30%-40%, 40%-50%, 50%-60%, 60%-70%, 70%-80%, 80%-90% and 90%-98% of the present compositions (w/w), or up to about 10%, 20%, 30%, 40%, 50%, 60%, 70%, 80%, 90% and 98% of the present compositions (w/w).

On the other hand, in numerous embodiments the sugars can constitute between about 10% to about 90% of the compositions of the invention (w/w), or more specifically between about 10%-20%, 20%-30%, 30%-40%, 40%-50%, 50%-60%, 60%-70%, 70%-80%, and 80%-90% of the present compositions (w/w), or up to about 10%, 20%, 30%, 40%, 50%, 60%, 70%, 80%, 90% of the present compositions (w/w).

With respect to additional components, in numerous embodiments the edible oil can comprise additional edible lipophilic substances, which can be single biological actives and combination of actives, complex extracts and superfoods.

In numerous embodiments the edible lipophilic substances can be selected from beneficial oils, nutraceuticals, vitamins, dietary or food supplements, nutrients, antioxidants, superfoods, natural extracts of animal or plant origin, probiotic microorganisms, or a combination thereof.

Examples of such combinations of edible oils and supplements are edibles oils with vitamins E or D, or combinations of lycopene and hemp oil exemplified in the present application. Lycopene is a powerful antioxidant with many health benefits, including the capability to improve heart health and lower risk of certain types of cancer. Hemp oil can play a crucial role in skin health and anti-aging.

The term ‘nutraceutical’ encompasses any edible lipophilic product with added health benefits, apart from nutrition. Examples of lipophilic nutraceuticals are fatty acids such as Omega 3, conjugated linoleic acid, butyric acid; carotenoids such as beta-carotene, lycopene, lutein, zeaxanthin; antioxidants such as tocopherols, flavonoids, polyphenols; and phytosterols such as stigmasterol, beta-sitosterol and campesterol.

The term ‘ [symbol] ’ herein broadly refers to a group of organic substances that are necessary in small quantities for normal health and growth in higher forms of animal life. Lipophilicity is a substantial problem with many important vitamins, such as vitamins A, D, E and K.

The term ‘ [symbol] ’ (also micronutrients) herein is a broad term which encompasses carbohydrates lipids, proteins, and vitamins. In terms of lipophilicity, notable examples are vitamins A, D, E and K, and carotenoids, with proven relevance to adipogenesis, inflammatory status, energy homeostasis and metabolism.

The term ‘ [symbol] ’ herein refers to any compound or combination of compounds that prevent oxidative stress. Notable examples of lipophilic antioxidants are tocopherols, flavonoids and carotenoids.

The term ‘ [symbol] ’ is a popular term for a food with superior nutrient density and health benefits. It is usually applied to certain types of berries, fish, leafy greens, nuts, whole grains, cruciferous vegetables, mushrooms, and algae and also olive oil and yogurt, in the natural form and in the form of extracts and dry matter.

The term ‘ [symbol] ’ encompasses herein any type of extracts from animal and plant sources, further including marine animals, particular types of mussels and marine phytoplankton that are considered superfoods.

The term ‘ [symbol] ’ encompasses herein any microorganism with benefits for the human microbiome, and specifically microorganisms of the genera: Lactobacillus, Bifidobacterium, Saccharomyces, Enterococcus, Streptococcus, Pediococcus, Leuconostoc, Bacillus, Escherichia coli.

The term ‘ [symbol] ’ herein relates to any product taken orally that contains one or more ingredients such vitamins, minerals, amino acids, and herbs or botanical extracts, or other substances that supplement human diet. It overlaps with the above groups, but it can further include additional substances, such as coenzyme Q10 which is an example of lipophilic dietary supplement.

It should be noted that the compositions of the invention can comprise more than one substance from the above groups and several groups of substances.

An extended list of edible polysaccharides and surfactants that relevant to the present compositions is provided in ANNEX A.

It should be noted that the compositions can comprise more than one candidate from these groups.

In the broadest sense, the relevant candidates to be included in the present compositions are substances regulated under GRAS and DSHEA that can be generally characterized as lipophilic.

As has been noted, in numerous embodiments, edible oil, per se, can characterized as nutraceuticals, vitamins, dietary supplements, nutrients, antioxidants and superfoods. One example of such oils is fish oil exemplified on this application.

Further, in numerous embodiments the present compositions can further comprise carriers, excipients, and additives for purposes of color, taste, and specific consistencies. The terms ‘ [symbol] ’ encompass herein any inactive substances that serve as the vehicle or medium for an active comprised in edible oil.

In numerous embodiments, the compositions can comprise coatings and package forms contributing to long term storage, stability, and other properties.

In numerous embodiments the compositions can comprise at least one carrier and/or at least one coating.

Gastro-resistant and controlled release coatings are especially applicable to oral dose forms, as they can protect and increase the effectiveness of actives. Such coatings can be achieved by various known technologies, such as the use of poly(meth)acrylates or layering. A well-known example of poly(meth)acrylate coating is EUDRAGIT®. Another important feature of poly(meth)acrylate coating is protection from external influences (moisture) or taste/odor masking to increase compliance.

The layering encompasses herein a range of technologies using substances applied in layers as a solution, suspension (suspension/solution layering) or powder (dry powder layering). Various characteristics can be achieved by adding suitable supplementary materials.

In other words, one of advantages of the present technology is its ability to provide a flexible product that can be adapted to various food technologies.

Another important feature of the compositions of the invention is an improved delivery of edible oils and lipophilic actives. The term ‘ [symbol] ’ encompasses herein improved solubility, absorption, or release of actives by any pharmacokinetic or pharmacodynamic parameters. Such properties have been presently exemplified.

The term ‘ [symbol] ’ encompasses herein a change in a range of about 5-10%, 10-15%, 15-20%, 20-25%, 25-30%, 30-35%, 35-40%, 45-50%, 50-55%, 55-60%, 60-65%, 65-70%, 70-75%, 75-80%, 80-85%, 85-90%, 90-95%, 95-100% relative to oil forms with the same actives, or up to 2, 3, 4, 5, 6, 7, 8, 9, 10, 20, 30, 40, 50, 60, 70, 80, 90, 100 fold relative to oil forms with the same actives.

Due to the particular structural properties of the compositions of the invention, the feature of improved delivery of actives further involves an immediate and/or a prolonged release to the GI tract, the circulation and/or tissues.

In other words, in certain embodiments the compositions of the invention can provide an immediate of edible lipophilic substances to a part of the GI tract, the plasma and/or one or more tissues.

The term ‘ [symbol] ’ implies that active can be measured in the GI or plasma within a relatively short period of time, such as after 1, 10, 20, 30, 40, 50, 60 min from the oral administration. It further implies a burst of active release with a subsequent decrease of the GI or plasma. The term further applies to the levels of active in organs or tissues (although with a slightly delayed timing), such as within 10, 20, 30, 40, 50, 60, 70, 80, 90 min from the oral administration thereof via oral or any other route.

In other embodiments the compositions of the invention can provide a prolonged delivery of edible lipophilic substances to a part of the GI tract, the plasma and/or tissues.

The term ‘ [symbol] ’ implies that active is measured in the GI, plasma and tissues with a lag, such as after 30, 60, 90, 120 min from the oral administration, and persists in the GI, plasma and tissues for 2 h, 3 h, 4 h, 5 h, 6 h, 7 h, 8 h and more after the oral administration.

In other embodiments the compositions of the invention can provide a biphasic release comprising an immediate and a prolonged delivery of edible lipophilic substances to a part of the GI tract, the plasma and/or tissues.

In certain embodiments the compositions of the invention provide immediate and/or prolonged release of edible lipophilic substances to the liver and brain.

The feature of improved delivery of oils and actives is directedly related to improved oral bioavailability. In numerous embodiments the compositions of the invention provide improved oral bioavailability of edible lipophilic substances compared to analogous oil forms. This feature has been presently exemplified with respect to various types of compositions of the invention.

In numerous embodiments the compositions of the invention provide an improved bio-accessibility of edible lipophilic substances compared to analogous oil forms. The term ‘ [symbol] ’ refers herein to a quantity of active released in the GI tract and becoming available for adsorption (e.g., enters the bloodstream), it is further dependent on digestive transformations of a compound into a material ready for absorption, the absorption into intestinal epithelium cells and the pre-systemic, intestinal, and hepatic metabolism. In other words, bio-accessibility reflects the degree of digestion in the GI.

Thus, in numerous embodiments the compositions of the invention can further provide an improved permeation of edible lipophilic substances into one or more parts of the GI tract compared to analogous oil forms.

In numerous embodiments the compositions of the invention can protect edible lipophilic substances from oxidation and degradation in the acidic pH of the stomach, and specifically in parts of the GI having a pH in a range between 1 to 7, or between 1 to 6, between 1 to 5, between 1 to 4, between 1 to 3 and between 1 to 2.

Especially with respect to supplements, nutrients and other actives, the features of improved delivery, oral bioavailability and bio-accessibility can further impact on effective dosing of actives, the number and frequency of consumption of actives and time to achieve a desired level physiological effect in a subject and to affect the general well-being of a subject overall.

Further, in numerous embodiments compositions of the invention can be adapted for oral, sublingual, or buccal administrations.

For supplements for example, in numerous embodiments such compositions can further comprise one or more types of coating, capsules or shells.

All of the above further apply to the methods, dosage forms and a variety of other applications to food industry.

More specifically, it is another objective of the invention to provide a dosage form comprising an effective amount of the compositions according to the above. This feature is particularly applicable to supplements and nutraceuticals comprising the dosage forms of the invention.

The term ‘ [symbol] ’ herein broadly relates to an amount or a concentration of active included in the composition or dosage form that was related in prior experience to a desired level physiological or clinically measurable response. An effective amount is further dependent on the number and frequency of administrations of the composition or dosage form. In the context of drugs and foods, an effective amount or concentration should comply with regulatory requirements such as FDA.

In numerous embodiments the dosage forms of the invention can further comprise a coating, a shell, or a capsule. These specific features have been discussed above.

In certain embodiments the coating, shell or capsule contribute to the prolonged delivery of the edible lipophilic substances comprised in the dosage forms.

In numerous embodiments the dosage forms of the invention can be adapted for oral, sublingual, or buccal administration.

In certain embodiments the dosage forms of the invention can be provided in a form of a sublingual patch. Specific patches using PVA have been presently exemplified. Sublingual patches can be produced from suitable plasticizing water dissolvable and non-toxic materials. Specific examples can include but, are not limited to, synthetic resins such as polyvinyl acetate (PVAc) and sucrose esters and natural resins such as resin esters (or ester gums), natural resins such as glycerol esters of partially hydrogenated resins, glycerol esters of polymerised resins, glycerol esters of partially dimerised resins, glycerol esters of tally oil resins, pentaerythritol esters of partially hydrogenated resins, methyl esters of resins, partially hydrogenated methyl esters of resins and pentaerythritol esters of resins, and further, synthetic resins such as terpene resins derived from alpha-pinene, beta-pinene, and/or d-limonene and natural terpene resins may be applied in the chewy base.

In numerous embodiments, the dosage forms of the invention can comprise a combination of lipophilic actives belonging to beneficial oils, nutraceuticals, vitamins, dietary or food supplements, nutrients, antioxidants, superfoods, natural extracts of animal or plant origin, probiotic microorganisms, or a combination thereof.

It is another objective of the invention to provide a method of making the presently described composition and dosage forms. The main steps in such method are:
i. Mixing at least one edible sugar, at least one edible polysaccharide, at least one edible surfactant, at least one edible oil and water
ii. emulsifying the mix to obtain a nanoemulsion,
iii. lyophilizing or spray dying the nanoemulsion.

The invention further provides a method for increasing loading of at least one edible lipophilic substance in an oral composition, the method comprising
(i) mixing an aqueous phase comprising at least one edible sugar, at least one edible polysaccharide and at least one edible surfactant with an oil phase comprising at least one edible lipophilic substance,
(ii) emulsifying the mix to obtain a nanoemulsion,
(iii) lyophilizing or spray dying the nanoemulsion.

Ultimately, it is one of the main objectives of the invention to provide the basis for making various foods, beverages and dietary products comprising the above-described compositions.

The terms ‘ [symbol] [symbol] ’ encompass herein a whole range of solid, semi-solid and liquid edible products, or orally consumable substances. These terms further encompass any type of sweets, chocolates, gums, and other forms of confection, and further, baked foods (such as biscuits, cakes, pies, cookies, pastries) and other chewable products.

In numerous embodiments the invention provides candies, lozenges, chewy candy products, bubble gums and other sweets comprising the above-described compositions.

In some embodiments the compositions of the invention can constitute up to about 0.001%, 0.005%, 0.01%, 0.05%, 0.1%, 0.5%, 1%, 5%, 10%, 20%, 30%, 40%, 50%, or more of the total solid or semisolid food (w/w).

Regarding beverages, the compositions of the invention are applicable to in any type of beverage, e.g., plain water, water-based liquids, alcoholic liquids, non-alcoholic liquids, juices, soft drinks, milk-based liquids, gaseous drinks, coffees, teas, etc.

In some embodiments the compositions of the invention can constitute up to about 0.001%, 0.005%, 0.01%, 0.05%, 0.1%, 0.5%, 1%, 5%, 10% of the total liquid (w/w).

The present application discloses several examples of methods of preparation of various food products. As a general method, the powder compositions of the invention can be either re-dispersed in water and mixed into foods and beverages or directly mixed into food and beverages, in any step of the production process.

In numerous embodiments the invention provides food supplements comprising the above-described compositions.

For this specific application, in some embodiments the compositions of the invention can constitute up to about 0.001%, 0.005%, 0.01%, 0.05%, 0.1%, 0.5%, 1%, 5%, 10%, 20%, 30%, 40%, 50%, 60%, 70%, 80%, 90% and 100% of the product (w/w).

In numerous embodiments the edible products can comprise additional materials for taste, coloring, and consistency, such as pectin, sugars, syrup, citric acid, sodium bicarbonate, etc. Use of such preparations is herein exemplified.

In certain embodiments the invention provides food additives comprising the above-described compositions.

In numerous embodiments the food additive can be a food colorant, a taste or an aroma enhancer, a taste masker, a food preservative, or a composition thereof. A non-limiting list of food additives that can be included in the compositions of the invention is provided in ANNEX A.

In the example of chewing gums, such products can further comprise gum base, softeners, sweeteners and flavorings. Known elastomer can include synthetic elastomers such as polyisobutylene, isobutylene-isoprene copolymer (butyl elastomer), styrene-butadiene copolymers, polyisoprene, polyethylene, and vinyl acetate-vinyl laurate copolymer; and natural non-degradable elastomers such as smoked or liquid latex, also guayule, jelutong, lechi caspi, massaranduba balata, sorva, perillo, rosindinha, massaranduba chocolate, chicle, nispero and gutta hang kang.

In some embodiments, the elastomer is Amylogum EST, the resin is Sisterna SP30, the softening compounds which is water insoluble is hard fat.

Additional gum additives can be one or more types of sweeteners, taste enhancers, flavoring agents, softeners, emulsifiers, coloring agents, acidulants, binding agents, fillers, antioxidants, and other components.

In certain embodiments, gum additives can include sugar, glucose syrup and sorbitol as sweeteners; Color GNT as a coloring agent; Flavor Bell Grape 6127832 as a flavoring agent; and lactic acid 88% as a softener

From another point of view, the invention provides compositions and dosage forms according to the above for use in improving the oral bioavailability of one or more edible lipophilic substances comprised in the respective compositions or dosage forms.

From yet another point of view, the invention provides compositions and dosage forms according to the above for use in improving the bio-accessibility of one or more edible lipophilic substances comprised in the respective compositions or dosage forms.

Still from another point of view, the invention provides a series of methods for improving the oral bioavailability and/or the bio-accessibility of one or more edible lipophilic substances in a diet of a subject, the main feature of such methods is administering to the subject an effective amount of the compositions and dosage forms according to the above.

Term ‘ [symbol] ’ encompasses herein any type of nutritional regimen.

In numerous embodiments the compositions and dosage forms of the invention can be administered together or separately from the subject's diet.

In other embodiments the compositions and dosage forms of the invention can be comprised in the subject's diet.

The invention can be further articulated in terms of use of the presently described compositions in the manufacture of foods, beverages, food additives or food supplements with improved oral bioavailability and/or improved bio-accessibility of edible lipophilic substances.

It should be noted that the compositions and dosage forms of the invention can assist in improving oral bioavailability of other diet ingredients, apart from those included in the compositions of the invention. In other words, they can serve as excipient foods in promoting the bioactivity of other substances.

There are new approaches to design edible compositions or structures of food matrixes to increase bioavailability that are giving rise to completely new classes of foods: functional foods, medical foods and excipient foods.

A [symbol] is produced from GRAS food ingredients, and typically contains one or more food-grade bioactive agents (‘nutraceuticals’) dispersed within a food matrix. There are already many examples of functional food products that are commercially available, including milks fortified with vitamin D, yogurts fortified with probiotics, spreads fortified with phytosterols, and breakfast cereals fortified with ω-3 fatty acids, vitamins, and minerals.

A [symbol] contains one or more pharmaceutical-grade bioactive agents (drugs) dispersed within a food matrix. This food matrix may be a traditional food type (such as a beverage, yogurt, or confectionary) or it may be a nutritional fluid that is fed to a patient through a tube. A medical food is usually administered to treat a particular disease under medical supervision. Medical foods are beyond the scope of this invention.

A new class of [symbol] is now being designed to improve the bioavailability of orally administered bioactive agents. An excipient food may not have any bioactivity itself, but it may increase the efficacy of any nutraceuticals or pharmaceuticals that are co-ingested with it. Some commonly used excipients in the pharmaceutical industry include lipids, surfactants, synthetic polymers, carbohydrates, proteins, cosolvents, and salts. Excipient foods are therefore meant to be consumed with a conventional pharmaceutical dosage form (e.g., capsule, pill, or syrup), a dietary supplement (e.g., capsule, pill, or syrup), or nutraceutical-rich food (e.g., fruits, vegetables, nuts, seeds, grains, meat, fish, and some processed foods). It is likely that different kinds of excipient foods will have to be designed for different types of bioactive agents. For example, the bio-accessibility of carotenoids in a salad may be increased by consuming it with a specifically designed salad dressing containing various food components that increase the bioavailability of the nutraceuticals in the salad: lipids that increase intestinal solubility; antioxidants that inhibit chemical transformations; enzyme inhibitors that retard metabolism; permeation enhancers that increase absorption; efflux inhibitors. Previous studies have shown that the bioavailability of oil-soluble vitamins and carotenoids in salads can be increased by consuming them with dressings containing some fat, which supports the concept of excipient foods.

Thus, the present technology makes part of the present effort to achieve functional and excipient foods.

A specific application of the present technology stems from finding and characterization an edible formulation of sugar with exceptionally fine particles and better rigidity, stability, sweetening capacity, dissolution rate and flow properties than the known sugar powders, and further, with the ability to control crystal size.

Essentially, the invention provides a sugar particle comprising a porous sugar material and lipophilic nanospheres having average sizes between about 50 to about 900 nm so that the lipophilic nanospheres are comprised within the porous sugar material, the sugar particle further comprises at least one edible sugar, at least one edible oil, at least one edible polysaccharide and at least one edible surfactant.

The term ‘ [symbol] ’ is meant to convey a solid sieve-like material with voids or pores which are not occupied by the main structure of atoms of the solid material (e.g., sugar). This term encompasses herein a material with regularly or irregularly dispersed pores, and pores in the form of cavities, channels, or interstices, with different characteristics of pores size, arrangement, and shape, as well as porosity of the material as a whole (the ratio of pores volume vs. the volume of solid material) and composition of solid material.

In certain embodiments, the porous sugar material can be characterized as a sugar scaffold. The term ‘ [symbol] ’ is meant to convey structural and functional properties, one of which is to contain or entrap the lipophilic nanospheres. The feature of entrapment of lipophilic nanospheres have been discussed in detail above.

In certain embodiments the lipophilic nanospheres can have an average size in the range between about 50-900 nm, and specifically in the range between about 50-100 nm, 100-150 nm, 150-200 nm, 200-250 nm, 250-300 nm, 300-350 nm, 350-400 nm, 400-450 nm, 450-500 nm, 500-550 nm, 550-600 nm, 650-700 nm, 700-750 nm, 750-800 nm, 800-850 nm, 850-900 nm and 900-1000 nm.

In certain embodiments the lipophilic nanospheres can have an average diameter in the range between about 100-200 nm, and specifically in the range between about 100-110 nm, 110-120 nm, 120-130 nm, 130-140 nm, 140-150 nm, 150-160 nm, 160-170 nm, 170-180 nm, 180-190 nm and 190-200 nm.

Thus, in numerous embodiments the size of the sugar particle can be in the range between about 10 μm and about 300 μm, and specifically in the range between about 10-50 μm, 50-100 μm, 100-150 μm, 150-200 μm and 250-300 μm or more.

In certain embodiments the size of the sugar particle can be in the range between about 20 μm to about 50 μm, and specifically in the range between about 10-50 μm, 20-50 μm, 30-50 μm, and 40-50 μm, or up to at least about 20 μm, 30 μm, 40 μm, 50 μm.

Within the indicated size ranges, in numerous embodiments the sugar particles of the invention can have an irregular shape or form (EXAMPLE 10).

The invention can be further articulated as edible formulations comprising a porous sugar material and lipophilic nanospheres having average sizes between about 50 nm to 900 nm, wherein the lipophilic nanospheres are comprised within the porous sugar material.

In numerous embodiments the formulations have a form of solid or semi-solid particles with size in the range between about 10 μm and 200 μm.

In other embodiments the formulations have solid or semi-solid particles with size in the range between about 20 μm and 50 μm.

One of the important features of the invention is that the size of sugar particles and the size of lipophilic nanospheres are correlated. While the size of sugar particles remains within a micronic range, it can be fined tuned or modified depending on the intensity of emulsification and the size of the lipophilic nanospheres (EXAMPLE 10.3).

As has been noted, the sugar particle is essentially composed of edible sugars, edible oils, edible polysaccharides, and edible surfactants. Characteristics of these components have been discussed in detail above.

The term ‘ [symbol] ’ encompasses herein short chain carbohydrates and sugar alcohols from natural and non-natural sources. A non-limiting list of applicable edible sugars is provided in ANNEX A.

In numerous embodiments the edible sugar is a natural sugar obtained from a vegetable or an animal source, a synthetic sugar, or a mixture thereof.

In certain embodiments the edible sugar can be obtained from a sugar beet, a sugar cane, a sugar palm, a maple sap and/or a sweet sorghum.

In certain embodiments the edible sugar can be lactose, a naturally occurring low sweet disaccharide produced by animals.

More generally, the applicable edible sugars are from natural sources, such as short chain carbohydrates and sugar alcohols.

In numerous embodiments the edible sugars are oligo-, di-, monosaccharides and polyols.

In certain embodiments, the edible sugars can be one or more mono- and/or disaccharides.

In further embodiments the edible sugar can be a mono- and/or a di-saccharide selected from glucose, fructose, sucrose, lactose maltose, galactose, trehalose, mannitol, lactitol or a mixture thereof.

In numerous embodiments the edible sugar can constitute between about 30% to about 80% of the sugar particle (w/w), or more specifically between about 20%-30%, 30%-40%, 40%-50%, 50%-60%, 60%-70%, 70%-80% and 80%-90% of the sugar particle (w/w).

The term ‘ [symbol] ’ encompasses herein hydrophilic polymers (hydrocolloids) of vegetable, animal, microbial, or synthetic origin with multiple hydroxyl groups, and may be polyelectrolytes. Certain examples are starch, carrageenan, carboxymethylcellulose, gum arabic, chitosan, pectin, and xanthan gum. A non-limiting list of the applicable polysaccharides is provided in ANNEX A.

In numerous embodiments the edible polysaccharides are selected from at least one of maltodextrin and carboxymethyl cellulose (CMC).

The term ‘ [symbol] ’ herein encompasses non-toxic edible nonionic and anionic surfactants, including among others cellulose ether and derivatives, citric acid esters of mono- and diglycerides of fatty acids (CITREM), diacetyl tartaric acid ester of mono- and diglycerides, various types of polyethylene sorbitol esters (Polysorbates, Tweens) and lecithins.

Under surfactants, in general, is meant emulsifiers and wetting agents. Common food emulsifiers are listed in ANNEX A.

In numerous embodiments the edible surfactants are selected from ammonium glycyrrhizinate, pluronic F-127 and pluronic F-68.

In other embodiments the edible surfactant can be a monoglyceride, a diglycerine, a glycolipid, a lecithin, a fatty alcohol, a fatty acid or a mixture thereof.

Still in other embodiments the edible surfactants can be selected from of a monoglyceride, a diglycerine, a glycolipid, a lecithin, a fatty alcohol, a fatty acid or a mixture thereof.

In certain embodiments the at least one edible surfactant is a sucrose fatty acid esters (sugar ester).

The term ‘ [symbol] ’ encompasses herein dietary saturated and unsaturated fatty acids, both from animal and plant sources. Under fats of animal origin is meant fats which are relatively high in saturated fatty acids, contain cholesterol and are usually solids at room temperature. Under fats or oils of plant origin is meant oils which are relatively high in unsaturated fatty acids (mono- or poly-unsaturated) and are usually liquid at room temperature. This term further encompasses exceptions such as tropical oils (e.g., palm, palm kernel, coconut oils), and partially hydrogenated fats, which are high in saturated fatty acids but remain liquid at room temperature because of high proportions of short-chain fatty acids. It further encompasses partially hydrogenated plant oils that are relatively high in trans fatty acids.

In numerous embodiments, the sugar particles can comprise more than one type of edible oil.

In numerous embodiments the edible oil is a natural oil obtained from a vegetable or an animal source, a synthetic oil or fat, or a mixture thereof.

Animal and vegetable oils and fats are predominantly mixtures of triglycerides.

In numerous embodiments the edible oil can comprise one or more triglyceride(s).

In numerous embodiments the edible oil is a solid (predominantly characteristic of oil from an animal source) and/or a liquid (predominantly characteristic of vegetable oils) at the ambient temperature.

The term ‘ [symbol] ’, or vegetable fats, encompasses herein oils extracted from seeds or other parts of plant fruits (in rare cases). A non-limiting list of edible vegetable oils is provided in ANNEX A.

In numerous embodiments the edible oils are selected from canola oil, sunflower oil, sesame oil, peanut oil, grapeseed oil, ghee, avocado oil, coconut oil, pumpkin seed oil, flaxseed oil, hemp oil, olive oil.

In numerous embodiments the edible oil can comprise Theobroma oil (cocoa butter).

The term ‘cocoa butter’ (also Theobroma oil) encompasses herein edible vegetable fats extracted from the cocoa beans characterized by specific flavor and aroma. It further refers to oils that are relatively abundant in stearic acid (C18:0), palmitic acid (C16:0) and oleic acid (C18:1), which is characteristic of cocoa butter. It further encompasses cocoa butter equivalents (CBE) characterized as two-thirds saturated fatty acids and one-third unsaturated fatty acid to meet the ratio typical of cocoa butter.

In numerous embodiments the sugar particles of the invention can further comprise one or more additional lipophilic actives.

In numerous embodiments the additional lipophilic actives can be selected from food colorants, taste or aroma enhancers, taste maskers, food preservatives.

The term ‘ [symbol] ’ herein encompasses four categories: (1) natural colors, (2) nature-identical colors, (3) synthetic colors, and (4) inorganic colors. It encompasses natural pigments and modification thereof, synthetic and inorganic colors.

The terms ‘ [symbol] ’ and ‘ [symbol] ’ broadly refers to compounds capable enhancing desirable tastes and odors, or alternatively compounds capable of reducing undesirable tastes (usually bitter, tasteless and sour). In some cases, the intrinsic components of the invention, i.e., the surfactants and polysaccharides, can act as taste maskers. Non limiting examples of taste enhancers and maskers are cyclodextrins, gelatin, gelatinized starch, lecithins or lecithin-like substances, and also camphor and terpen derivatives such as fenchone, borneol and isoborneol.

The term ‘ [symbol] ’ broadly refers to food additives that reduce the risk of foodborne infections, decrease microbial spoilage and preserve freshness and nutritional qualities of foods. Acidulants, organic acids and parabens are often used as antimicrobials, alone or in conjunction with antioxidants.

A non-limiting list of relevant substances is provided in ANNEX A.

In numerous embodiments the additional lipophilic active can be selected from beneficial oils, nutraceuticals, vitamins, dietary or food supplements, nutrients, antioxidants, superfoods, natural extracts of animal or plant origin, probiotic microorganisms, or a combination thereof. Candidate actives and agents belonging to these groups have been discussed in detail above.

Ultimately the invention provides a food product comprising the specified sugar particle or the edible formulation thereof.

The terms ‘ [symbol] ’ or ‘ [symbol] ’ refer herein to foods, beverages and dietary products. They encompass herein a whole range of consumable substances, including any type of sweets, bakery products, soft and alcoholic beverages, etc. They further relate to sweetened food supplements, nutrients and other health beneficial additives.

Thus, in certain embodiments the invention provides foods or food products comprising a plurality of sugar particles according to the above.

In other embodiments the invention provides beverages comprising a plurality of sugar particles according to the above.

The invention is particularly applicable to chocolate and bakery products, which require particular size and texture of sugar.

Thus, in certain embodiments the applicable food products can be, but are not limited to, baked foods (such as biscuits, cakes, pies, cookies, pastries), chocolates, gums, mints, lozenges, jellies, hard candies, soft candies, gummies, truffles, caramels, taffy, nougat and other chewable products.

In numerous embodiments, the foods and beverages can comprise additional materials for taste, coloring, and consistency, such as pectin, sugars, syrup, citric acid, sodium bicarbonate, etc.

For specific purposes, such as nougat for example, the product can comprise additional substances such as egg albumin, hard fat, flavoring powders (e.g., milk powder, cocoa powder and fondant powder) and other additives.

In certain embodiments the invention provides food additives comprising a plurality of sugar particles according to the above. Characteristics of such additives have been discussed above.

In numerous embodiments the invention provides supplements comprising a plurality of sugar particles according to the above. Candidate actives belonging to this group have been discussed in detail above.

In some embodiments the sugar particles of the invention can constitute up to about 1%, 2%, 3%, 4%, 5%, 6%, 7%, 8%, 9%, 10% of the food product (w/w).

The low concentrations are especially applicable to beverages.

In further embodiments, the sugar particles of the invention can constitute up to about 20%, 30%, 40%, 50%, 60%, 70%, 80%, 90% and 100% of the edible product. Higher concentrations are especially applicable to sweets and food supplements.

In yet other embodiments the invention provides delivery systems comprising a plurality of sugar particles according to the above. As has been noted, many researchers and industries are currently developing various delivery systems to increase the oral bioavailability of lipophilic bioactive agents. There are significant challenges associated with incorporating different bio-actives into foods, beverages, and other consumable forms for creating new excipient and functional foods.

This aspect can be further articulated in terms of use of the sugar particles according to the above in the manufacture of sweetened food and beverage products or sweetened supplements.

Ultimately, the invention provides a method for preparing the sugar particles with the particle size in the range between about 10 μm and about 300 μm. The main steps of this method are:
mixing at least one edible sugar, at least one edible polysaccharide, at least one edible surfactant, at least one edible oil and water
emulsifying the mix to obtain a nanoemulsion,
lyophilizing or spray dying the nanoemulsion.

The term “about” in all its appearances in the text denotes up to a ±10% deviation from the specified values and/or ranges, more specifically, up to ±1%, ±2%, ±3%, ±4%, ±5%, ±6%, ±7%, ±8%, ±9% or ±10% deviation therefrom.

### EXAMPLES

Any methods and materials similar or equivalent to those described herein can be used in the practice or testing of the present invention. Some embodiments of the invention will be now described by way of examples with reference to respective figures.

### Example 1: Powder Composition with Edible Oils

### 1.1 Preservation of Nanospheres Size in Reconstituted Compositions

A powder composition comprising 30% of AlaskaOmega (Omega 3) was prepared by nano-emulsification, freezing in liquid N₂ and lyophilization (48 h). Particle size, distribution and uniformity was evaluated after nano-emulsification and lyophilization upon dispersion of the powder in TWD to 1% (w/w), using PDI (poly dispersity index) measured by DLS (dynamic light scattering). Measurements were perfumed in triplicates. PDI correlates to particle size.

The PDI results suggested that the nanoemulsion and the reconstituted powder yielded a uniform and homogenous population of particles with the average size of 149 nm±SD for the nanoemulsion and 190 nm±SD for the reconstituted powder. The differences between samples were insubstantial.

The results suggest that upon reconstitution in water, the powder compositions of the invention preserve the particle size compared to the source nanoemulsion, and that this feature is relatively uniform and homogeneous in the sample, overall.

Preservation of particle size in powders reconstituted in water solutions is further indicative of the same trend in saliva and the GI.

1.2 Preservation of Nanospheres Size after Storage for 1 Month

The powders were stored for 1 month, and then reconstituted in TWD to 1% (w/w) or to 2% (w/w) and subjected to DLS or Cryo-TEM (transmission electron cryo-microscopy) analyses, respectively.

As per DLS, the average particle size in the reconstituted powder was 218 nm±SD. As per Cryo-TEM, the average size was 100 nm±SD. The two technologies yielded certain differences.

Overall, the results suggest that the powder compositions have high stability, while preserving the reconstitution capacity to a uniform, homogenous and nanometric particle size.

1.3 Powder Compositions with Lycopene Oil and Hemp Oil

Analogous experiments were conducted with powder compositions comprising a combination of lycopene oil and hemp (1:1.4, respectively). Powders were produced as in 1.1. DLS analysis was performed on the nanoemulsion and the reconstituted powder (1% w/w).

DLS analysis showed a population of particles in the nanoemulsion with the average size of about 590 nm and two populations of particles in the reconstituted powder with the average size of about 272 nm and a minor peak at 79 nm. It should be noted that the particle size was not increased after lyophilization.

The results suggest that the powder compositions with lycopene and hemp oils behave similarly to the powder with Omega 3 in terms of preservation of particle size, uniformity, and homogeneity. Overall, the results suggest that the technology is adaptable to various types of edible oils and combinations of oils.

### 1.4 Stability Studies in Compositions Comprising Cannabinoids

Powder compositions comprising cannabinoid (CBD or THC) were stored at 45° C. (oven) for 1, 35, 54, 72 and 82 days (3 months correlates to 24 months at RT). Particle size was evaluated using DLS. The results are shown in Table 1 and FIG. 1.

TABLE 1
DS measurements in the test samples

| Temp | AVG | PDI | PEAK |
|---|---|---|---|
| RT | 150.5 | 0.208 | 163.2 |
| 1 day at 45° C. | 149.1 | 0.213 | 151.9 |
| 35 days at 45° C. | 160.2 | 0.25 | 159.6 |
| 54 days at 45° C. | 150.1 | 0.216 | 144.7 |
| 72 days at 45° C. | 150.1 | 0.212 | 143.3 |
| 82 days at 45° C. | 153.7 | 0.205 | 154 |

AVG average diameter (nm)
PDI polydispersity index

The results show that the nanospheres size was preserved for at least three months at 45° C., thus suggesting that the powder compositions have long-term stability and ability to preserve particle size upon reconstitution in water solutions or the GI.

1.5 Compositions with Lactose and Hemp Oil

Nanoemulsions were prepared with the constituents detailed in Table 2 using lactose as a choice of sugar.

TABLE 2
Specifications of the test samples

| Lactose | 80% | 90% | 100% | 110% | 120% |
|---|---|---|---|---|---|
| Ammonium Gly | 3.05 | 3.05 | 3.05 | 3.05 | 3.05 |
| Meltodextrin | 13.68 | 13.68 | 13.68 | 13.68 | 13.68 |
| Lactose | 16 | 18 | 20 | 22 | 24 |
| Water | 145.74 | 145.74 | 145.74 | 145.74 | 145.74 |
| Hemp oil | 15.74 | 15.74 | 15.74 | 15.74 | 15.74 |

Nanoemulsions were prepared from a solution of lactose (80%) and maltodextrin (25-50° C.). Lactose was added to various concentrations of 80%, 90%, 100%, 110%, 120%. Ammonium Gly and hemp oil were added as per the amounts in Table 2. The preparations were homogenized by (M-110EH-30) at 10,000-20,000 PSI (25-50° C.)×4. Powders were prepared by: (1) lyophilization, whereby the nanoemulsions were frozen (−25 to −86° C.) and lyophilized (12-24 h, −51° C., 7.7 mbar); (2) spray drying, whereby nanoemulsions were pumped with peristaltic pump (rate 8.5-20 g/min, air temp. 110-150° C., air flow 0.4-0.5 m³/min, atomizer pressure 0.15 MPa).

DLS analysis of the reconstituted powders is shown in Table 3.

TABLE 3
DS measurements in the test samples

| Lactose | Drying | | T air | Pump rate | Average Size |
|---|---|---|---|---|---|
| conc. | technology | Yield (%) | out | (g/min) | (nm) |
| 80% | Spray dryer | 54.8 | 62 | 8.78 | 135.3 |
| 90% | Spray dryer | 63.8 | 62 | 9.66 | 127.6 |
| 100% | Spray dryer | 87.5 | 63 | 10.4 | 125.6 |
| 120% | Spray dryer | 87 | 63 | 10.1 | 124.6 |
| 80% | Lyophilizer | 100% | NR | NR | 136.1 |
| 100% | Lyophilizer | 100% | NR | NR | 127.8 |
| 110% | Lyophilizer | 100% | NR | NR | 125.4 |
| 120% | Lyophilizer | 100% | NR | NR | 124.5 |

The results suggest preservation of nanospheres size under various manipulations, and with various concentrations of lactose. Overall, the results suggest that lactose can serve as an alternative sugar without disrupting the core properties of the composition.

### 1.6 Loading Capacity and Distribution of the Oil Component

Nanoemulsions were prepared with various types of edible oils: Omega 7, TG400300, EE400300. Surface oil content was determined by hexane. Powders (5 g) were washed with hexane (50 ml), filtered, and washed (×4) with hexane (5 ml). Loss on drying (LOD) was performed on the filtrate under stream of N₂ until stabilization of weight. The oil content inside the nanospheres was estimated as:
Omega 7—52.67%
TG400300—30.67%
EE400300—35.33%

The results suggest that up to about 50% oil can be incorporated into the lipophilic nanospheres, depending on the type of oil (e.g., Omega 7 vs. TG400300 and EE400300). The result is indicative of a comparable distribution of lipophilic active(s).

The results further suggest that a substantial proportion of oil can be present outside the nanospheres. This finding strongly supports the notion of differential bioavailability and biphasic release of the oil and the entrapped actives as revealed in studies in vivo in EXAMPLE 3.

As per current studies, up to 80% oil can be incorporated into the nanospheres.

Overall, these results are indicative of high loading capacity of the compositions with respect edible oils and lipophilic actives.

### 1.7 Encapsulation Capacity of the Compositions

Encapsulation efficiency was estimated by the difference between the initial amount of active added and the amount unentrapped in the composition. Four different types of powders were prepared with the following actives using the same procedure:
Vitamin D3 oil
Passionfruit oil
Medium-chain triglyceride (MCT) oil
Pomegranate seed oil

The encapsulated oil was determined after removing the non-encapsulated oil component with hexane (shaking 1 g powder in 10 ml n-Hexane for 2 min). The product was filtered (over Watman and vacuum) and washed with hexane (×3), and the oil content was measured using Solvent extraction-gravimetric method. The results are shown in Table 4.

TABLE 4
The entrapped oil content in the tested compositions

| | Before wash | After wash | Encapsulation |
|---|---|---|---|
| Oil/active | (gr/100 gr) | (gr/100 gr | efficiency |
| Vitamin D | 30.57 | 30.50 | 99.8% |
| Passion fruit | 30.31 | 29.46 | 97 2% |
| MCT | 29.06 | 28.79 | 99.1% |
| Pomegranate | 29.16 | 26.11 | 99.8% |

The results suggest a substantially highly loading capacity of lipophilic actives into the particulate matter of the compositions of the invention. The loading capacity characteristic of the compositions of the inventions is in the range of 97.0-99.8%.

### Example 2: Powders with Supplements and Extracts

2.1 Compositions with Korean Ginseng and Preservation of Particle Size

Red Korean Ginseng oily extract (6 years old) was formulated using the technology of the invention: (1) by the production of a nanoemulsion and (2) a drying process (Ginseng oil/fixed oil, 1:2, 30% oil in powder). Particle size was determined in the nanoemulsion and in reconstituted powder as above.

DLS analysis showed that the populations of particles in the nanoemulsion and the reconstituted powder were similar by size (about 163 nm and 180 nm, respectively), and did not increase during the production process.

2.2 Compositions with Additional Lipophilic Oils

The following powders were prepared using the above methods:
Sample 1—Fish oil FO 1812 Ultra, 50% oil
Sample 2—KD-PUR 490330 TG90 Ultra, 30% oil
Sample 3—KD-PUR 490330 TG90 Ultra, 50% oil

Particle size was evaluated in the nanoemulsions and the reconstituted powders as above. The particle size remained surprisingly stable, among samples and in the respective nanoemulsions and reconstituted powder, with an average size ranging from about 140-160 nm.

In summary, the different compositions showed consistency of particle size in the transition from nanoemulsion to solid forms. The particles size remained stable during the drying process, which was highly surprising. This experiment suggests high applicability of the technology for numerous lipophilic nutraceuticals and supplements.

2.3 Compositions with High Content of Oils and Lipophilic Actives

Curcumin 70%

| Ingredient | amount (gr) |
|---|---|
| Sucrose | 9.1 |
| Maltodextrin | 6.1 |
| Ammonium Gly | 2.8 |
| Curcumin extract powder | 42 |
| Water | 140 |

Sucrose and Maltodextrin were fully dissolved in water. Curcumin powder was dry blended with Ammonium Gly and the solution was added until homogenous emulisification. The emulsion was fed to the microfluidizer (4 bar, 16,000 PSI, ×2 cycles).

Q10 100%

| Ingredient | amount (gr) |
|---|---|
| Ammonium Gly | 4 |
| Q10 | 56 |
| Water | 140 |

Q10 powder was dry blended with Ammonium Gly, mixed and homogenized with water until homogenous emulsification. The emulsion was fed to the microfluidizer

(4 bar, 16,000 PSI, ×2 cycles).

CBD and MCT 70% Oil

| Ingredient | amount (gr) |
|---|---|
| Sucrose | 7.6 |
| Maltodextrin | 5 |
| Ammonium Gly | 2.4 |
| Glycerin | 3 |
| CBD | 21 |
| MCT | 21 |
| Water | 140 |

CBD was dissolved in MCT at 40° C. and Ammonium Gly was added until even dispersion. Sucrose, Maltodextrin and Glycerin were dissolved in the water. The mixture of oil and active was added to the sugar solution, mixed, and homogenized until smooth emulsification. The emulsion was fed to the microfluidizer (4 bar, 16,000 PSI, ×2 cycles).

### Example 3: Formulations in a Form of Sublingual Patch

The experiment explored application of the technology to PVA sublingual films. To that end, powders containing 30-50% oil were reconstituted in TDW to 5% (w/w). PVA solution (4.5%) was prepared from PVA powder (86-89 hydrolyzed PVA) in TDW. The PVA solution was mixed with the nanoemulsion in proportions 4% and 0.5%, respectively. Samples of the mix (3 gr) were casted into aluminum mold (6 samples) and were dried at 38° C. for 24 h. Some samples included flavoring agents. Specifications are further detailed in Table 5.

TABLE 5
Specifications of the sample

| | Nanoemulsion | Actual PVA | Sample size | |
|---|---|---|---|---|
| #sample | addition (g) | conc. | (g) | Dry weight (g) |
| 1 | | 8.0% | 2.5 | 0.20 |
| 2 | 2.1 | 7.6% | 4.2 | 0.34 |
| 3 | 2.1 | 7.3% | 4.2 | 0.32 |
| 4 | 2.1 | 6.9% | 4.2 | 0.31 |
| 5 | 2.1 | 6.6% | 4.2 | 0.28 |
| 6 | 2.1 | 6.3% | 4.2 | 0.30 |

All samples produced films, the observed differences in shape were probably due to different wetting properties. Table 6 shows comparison between the actual dry weight and theoretical weight, suggesting a complete evaporation of water during drying. The nanoemulsion was uniformly dispersed across the films.

TABLE 6
Estimates of the actual and theoretical weight

| | Nano-particles | | |
|---|---|---|---|
| PVA theoretical | theoretical | Total theoretical | Actual dry |
| content (g) | content (g) | dry compounds (g) | weight (g) |
| 0.20 | 0.000 | 0.20 | 0.20 |
| 0.32 | 0.008 | 0.33 | 0.34 |
| 0.31 | 0.017 | 0.32 | 0.32 |
| 0.29 | 0.025 | 0.32 | 0.31 |
| 0.28 | 0.034 | 0.31 | 0.28 |
| 0.26 | 0.042 | 0.31 | 0.30 |

Selected samples (N=3) were dissolved in 50 ml TDW at 37° C. for 20-40 min to produce solution. Sample 6 (dry weight 0.15 g) was analyzed for oil content and was identified with about 0.017 g oil—83.6% of theoretical content.

The produced film (1*1 cm², ˜100 μm thick) was placed under the tongue, the time to complete dissolution was measured.

The results suggest that the powder was suitable for formulation in sublingual films. The solid particles were evenly fixed in the polymerized film to create a solid-in-solid dispersion. Upon dissolution, the particles were completely released from the polymer matrix.

Overall, a sub-lingual film provides an attractive approach for delivery of lipophilic supplements and nutrients.

### Example 4: Surprising Chemical Stability of Actives

### 4.1 Stability of Compositions Comprising Cannabis Extracts

Cannabinoids are especially prone to chemical and photolytic degradation. Nanoemulsions were prepared with full spectrum Cannabis oil (50%) from two Cannabis strains (THC or CBD enriched), and the other core components of the compositions of the invention. The reconstituted powders yielded the characteristic particle size of 150 nm and the original cannabinoid spectrum in oil. Powders were stored in aluminum bags in the following conditions in 40° C. chamber:
1 gr per bag
O₂ scavenger
Silica humidifier

The experiment was performed in two independent runs for products from THC and CBD enriched strains (Powder A and Powder B) Cannabinoid analysis was performed at Baseline (0), 30 days, 45 days, and 83 days (correlates to 10, 13, 24 months under standard conditions) using HPLC. The results are shown in Tables 7 and 8.

TABLE 7
Cannabinoid analysis in Powder A

| Analyte content | | | | | Total |
|---|---|---|---|---|---|
| (% w/w) | THC-Δ-9 | CBD | CBG | CBN | cannabinoids |
| T0 | 2.71 | 1.05 | 0.09 | 0.08 | 3.93 |
| 10 months | 2.62 | 1.03 | 0.09 | 0.09 | 3.83 |
| 13 months | 2.68 | 1.02 | 0.07 | 0.09 | 3.86 |
| 24 months | 2.62 | 1.03 | 0.09 | 0.09 | 3.83 |

TABLE 8
Cannabinoid analysis in Powder B

| Analyte content | | | | | Total |
|---|---|---|---|---|---|
| (% w/w) | THC-Δ-9 | CBD | CBG | CBN | cannabinoids |
| T0 | 0.28 | 3.95 | 0.01 | 0.07 | 4.31 |
| 10 months | 0.28 | 3.98 | 0.01 | 0.02 | 4.29 |
| 13 months | 0.27 | 3.93 | 0.01 | 0.09 | 4.3 |
| 24 months | 0.28 | 3.98 | 0.01 | 0.02 | 4.29 |

The results suggests that the compositions of the invention provide long-term stability for cannabinoids and complex compositions of cannabinoids from natural origin, such as Cannabis oil extracts, for at least 24 months at RT. The recommended storage conditions are in aluminum bags with O₂ scavenger and/or moister desiccator.

Under these conditions, the maximum degradation rate did not exceed 2.5% for the entire cannabinoid content and was even lower for specific cannabinoids, i.e., THC and CBD as CBN and CBG. This finding is further consistent with the stability of CBN (in Powder A for example) as a known marker of cannabinoid degradation.

### 4.2 Stability of Compositions Comprising Lycopene

Carotenoids are known to be sensitive to increased temperature, pro-oxidative species, and acidic pH. Nanoemulsions were prepared with lycopene oleoresin (6% lycopene w/w) and the other core components of the compositions of the invention. Powders (4 gr) were heat-sealed with vacuum in aluminium bags with moister and oxygen scavengers, and stored for 0, 30, and 90 days at RT (25° C.), 4° C., and 40° C. (in duplicates). Products were tested by visual appearance, DLS and HPLC analyses at the Baseline and storage time points.

Visual analysis suggested that all samples preserved a typical texture, confluence, and color over the storage period. DLS analysis did not reveal any significant deviations from the original particle size of 225-272 nm. The results are shown in Table 9.

TABLE 9
DLS analysis of compositions with lycopene

| Storage temperature | Time 0 | Time 30 days | Time 90 days |
|---|---|---|---|
| RT (about 25° C.) | 260 nm | 225 nm | 236 nm |
| 4° C. | | 272 nm | 265 nm |
| 40° C. | | 246 nm | 251 nm |

Similarly, HPLC analysis showed only minimal losses of lycopene over the storage period as 7%, 3%, and 1% for samples stored at RT, 4° C., and 40° C., respectively.

Overall, the results suggest that the compositions of the invention provide an extended shelf life for lycopene and prevents its degradation. The recommended packing includes an aluminum bag with moister and oxygen scavengers. The findings of extended stability for 90 days at 40° C. are correlative to 2-years at RT.

4.3 Stability of Compositions with Vitamin D3

Analogous analysis was performed for powders comprising vitamin D3 under storage conditions of 40° C./RH 75° C. for 90 days. Products were analyzed by HPLC regarding vitamin D and ethoxy vitamin D degradation product. Analytical tests were performed in-house and validated by an external authorized laboratory (Eurofins). The results are shown in Table 10.

TABLE 10
HPLC analysis of compositions with vitamin D3

| | | Vitamin D | |
|---|---|---|---|
| | | degradation | |
| | Vitamin D | product | Eurofins results |
| Vitamin D3 oil | 24.14 mg/gr | 0.70 mg/gr | 26.3 mg/gr |
| Vitamin D3 powder | 6.76 mg/gr | 0.40 mg/gr | 7.7 mg/gr |
| Day 1 | | | |
| Vitamin D3 powder | 6.60 mg/gr | 0.47 mg/gr | Duplicate 1 - |
| Day 90 | | | 6.9 mg/gr |
| | | | Duplicate 2 - |
| | | | 7.4 mg/gr |

The cholecalciferol tests of vitamin D3 oil were verified and found to be consistent with its certificate of analysis of 1M iu/g.

The results suggest that 28%-29% of vitamin D3 oil was encapsulated. Since the composition was prepared with 30% oil, this result indicates minimal losses during the production process.

The results further suggest minimal cholecalciferol degradation of up to 5%. The differences between the duplicates can derive from the soldering quality. Further, despite that the powder was kept at accelerated conditions (40° C. and 75% R.H versus 4-8° C.), it had far fewer degradation products compared to oil. Overall, the experiment indicates product stability over 2 years at RT.

The above studies suggest that the powder compositions of the invention have surprising capability to preserve actives over prolonged period, in other words, an accelerated chemical stability and prolonged shelf-life. This feature is surprising, especially in view that the production process involves high pressure, water environment, both of which are unfavorable for lipophilic molecules, and further in view that the reduction of particle size and the subsequent increase in the particle surface area are expected to increase actives oxidation and chemical instability.

These findings further support applicability of the powder compositions of the invention for producing of various types of food products and food additives.

4.4 Stability of Compositions with Fish Oil

Another study explored the protective property of the powder compositions with a fish oil. Fish oils (60% Omega 3 fatty acids w/w) oxidize readily, forming primary and secondary oxidation products, which may be harmful for humans.

The powder compositions were prepared from 40% fish oil (w/w and the other core components of the compositions of the invention.

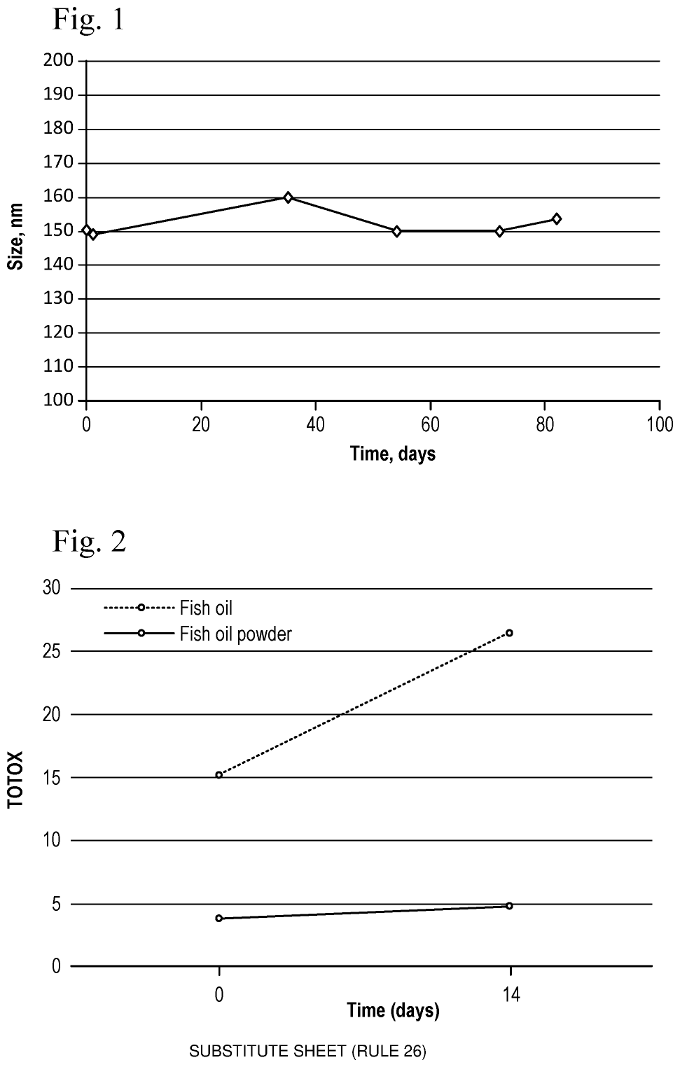

The oil and powder samples were exposed to environmental oxygen, and then heat-sealed with vacuum and stored at 4° C. for 28 days. The primary (peroxide; PV) and the secondary (anisidine; AV) oxidation products were measured at days 0, 14, and 28. TOTOX value (overall oxidation state) was calculated by Formula: TOTOX=AV+2*PV. The results are shown in FIG. 2.

The results show that the powder composition a significantly lower TOTOX, i.e., a significantly lower concentrations of primary and secondary oxidation products, compared to the oil form starting from day 0 and even after 14 days. The result of day 0 is particularly interesting since the production process of the powder includes exposure to water and oxygen.

Overall, the results point to a surprising protecting capacity of the powder compositions, most likely due to the unique property of encapsulation of actives and prevention of exposure and consequent oxidation and degradation oxidation-sensitive lipids comprised in the fish oil. This property is further consistent with the previously demonstrated long-term stability characteristic of the present powder compositions.

### Example 5: Surprising Loading Capacity

The study explored loading capacity of the powder compositions of the invention with the example of concentrated Cannabis oil. Nanoemulsions were produced with raw RSO high THC concentrate (1 gr) by the above methods. The nanoemulsions and the reconstituted powders yielded particles with characteristic size of about 150 nm. The reconstituted powders were subjected to analysis of cannabinoids using HPLC. Table 11 shows comparison between the calculated vs. actual cannabinoid content.

TABLE 11
Comparison between the measured and calculated THC content

| % w/w | Calculated | Measured |
|---|---|---|
| Δ9-THC | 8.945% | 8.45% |
| CBG | 0.276% | 0.24% |

The ratio between the calculated and actual content of A9-THC is 94.91%.

The ratio between the calculated and actual content of CBG is 86.9%.

The results point to surprisingly high capacity of loading of oils and actives into the compositions of the invention as reflected in the proportion of oil relative to the total powder material.

### Example 6: Surprising Bioavailability Profiles

Oral bioavailability of compositions of the invention was evaluated in a rat model. The study compared two prototype compositions of cannabinoids (CBD/THC), an oil composition (LL-OIL) and a powder composition (LL-P with regard to actives release in plasma and selected organs. The study used the following variables and end points:
i. Mortality and morbidity monitoring—daily.
ii. Body weight monitoring—during acclimation and before dosing.
iii. Clinical observation—prior to and for 2 h after
iv. Blood draws—at timepoints of 0, 15, 30, 45, 60, 90, 120 and 240 min.
v. Termination and organ collection (brain, liver) at 45, 60, 90, 120, 240 min.

The study used classic procedures for pharmacokinetic (PK) and biodistribution analyses Animals (N=12) were divided into 2 groups as per LL-OIL and LL-P.

Materials and Methods
Test item I: CBD/THC POWDER (LL-P): LL-CBD-THC 30% OIL in powder
Test item II: CBD/THC OIL (LL-OIL): LL-CBD-THC OIL diluted in hemp oil

For LL-P, an oral dose of 225 mg of the powder was dissolved in 4.275 mg TDW and administrated per rat. For LL-OIL, an oral dose of 67.5 mg of the oil was diluted in 1 mL hemp oil and administered per rat.

Male/12/376/456 g (sex/number/weight) SD rats divided into groups (deviation of ±20% from mean weight in each group) and acclimatized (8 days). For the entire study there were no findings of morbidity, prolonged pain or distress.

The study was conducted in 1 cycle for 2 groups (N=6 per group, 3-4 time points per animal). Test items were administered to 6 animals with subsequent bleeding and termination at time points as in Table 12.

TABLE 12
Group allocation

| | | Dose | | | | | | |
|---|---|---|---|---|---|---|---|---|
| | Dose | volume | | | | | | |
| Group | (mg/kg) | (ml/kg) | Route | Animal | Bleeding time point | | Termination | |
| LL-P | THC | 10 | Oral | 1 | 0, 15, 45 | min | 45 | min |
| | 13.5 | | | 2 | 0, 30, 60, 240 | min | 240 | min |
| | CBD | | | 3 | 15, 45, 60 | min | 60 | min |
| | 15.7 | | | 4 | 30, 60, 90 | min | 90 | min |
| | | | | 5 | 45, 90, 120 | min | 120 | min |
| | | | | 6 | 0, 15, 30, 90 | min | 90 | min |
| LL- | | 3 | | 7 | 0, 15, 45 | min | 45 | min |
| OIL | | | | 8 | 0, 30, 60, 240 | min | 240 | min |
| | | | | 9 | 15, 45, 60 | min | 60 | min |
| | | | | 10 | 30, 60, 90 | min | 90 | min |
| | | | | 11 | 45, 90, 120 | min | 120 | min |
| | | | | 12 | 0, 15, 30, 90 | min | 90 | min |

Body weight was recorded at the initiation of the study. Animals were observed daily for toxic/adverse symptoms before and after administration. Blood samples were collected at Baseline and after administration at the indicated time-points and stored. Organs were collected from animals after terminal bleeding and perfusion, selected organs (brain, liver) were collected and stored at −80° C. Variations in organs weight were insignificant.

Results

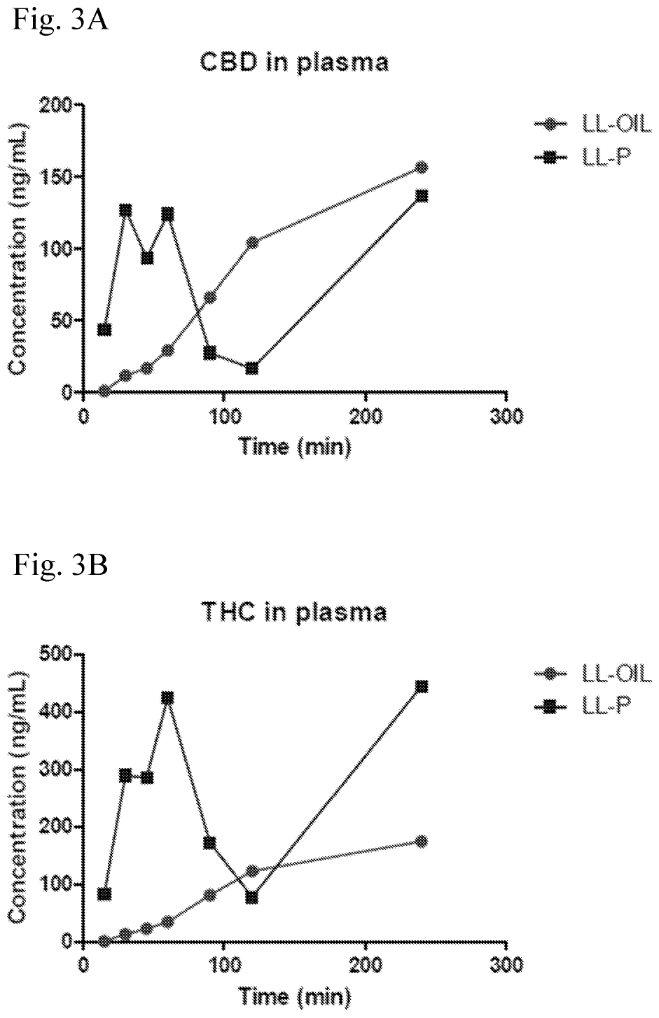

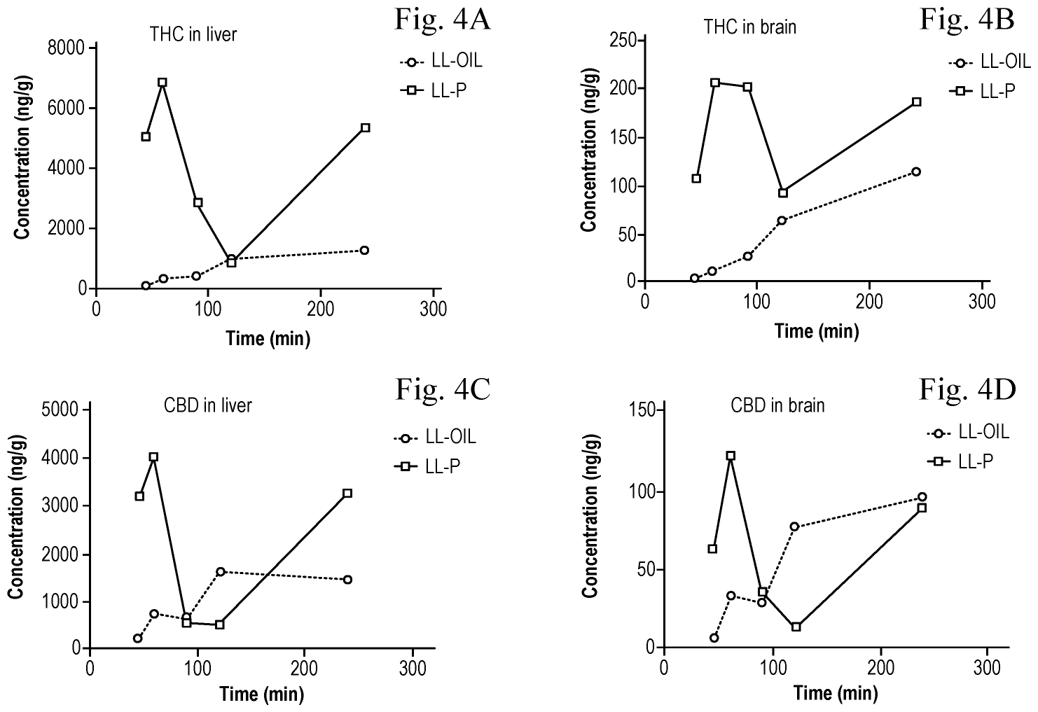

Pharmacokinetic (PK) analysis of CBD and THC for both test items in plasma, brain and liver is presented in Table 13. Plasma concentrations of CBD and THC in the two groups are shown in FIGS. 3A-3B. Tissue distribution (liver and brain) of CBD and THC in the two groups are shown in FIGS. 4A-4D.

TABLE 13
PK analysis of CBD and THC in plasma, brain and liver

| | | CBD | CBD | THC | THC |
|---|---|---|---|---|---|
| | | LL-P | LL-OIL | LL-P | LL-OIL |
| General PK parameters: | | PLASMA | PLASMA | PLASMA | PLASMA |
| Dose Amount | mg | 6.5 | 6.5 | 5.6 | 5.6 |
| Dosage | mg/kg | 15.7 | 15.7 | 13.5 | 13.5 |
| Cₘₐₓ (obs) | ng/ml | 137.0 | 156.6 | 444.4 | 174.6 |
| Tₘₐₓ (obs) | hr | 4.0 | 4.0 | 4.0 | 4.0 |
| AUC (0-4) (obs area) | ng-hr/ml | | | | |
| | | THC | THC | CBD | CBD |
| | | LL-P | BRAIN | LL-P | BRAIN |
| General PK parameters: | | BRAIN | LL-OIL | BRAIN | LL-OIL |
| Dose Amount | mg | 5.6 | 5.6 | 6.5 | 6.5 |
| Dosage | mg/kg | 13.5 | 13.5 | 15.7 | 15.7 |
| Cₘₐₓ (obs) | ng/g | 206.9 | 115.0 | 122.6 | 95.6 |
| Tₘₐₓ (obs) | hr | 1.0 | 4.0 | 1.0 | 4.0 |
| AUC(0-4) (obs area) | ng-hr/g | 536.5 | 215.0 | 201.0 | 221.5 |
| | | THC | THC | CBD | CBD |
| | | LL-P | LL-OIL | LL-P | LL-OIL |
| General PK parameters: | | LIVER | LIVER | LIVER | LIVER |
| Dose Amount | ng | 5.6 | 5.6 | 6.5 | 6.5 |
| Dosage | ng/kg | 13.5 | 13.5 | 15.7 | 15.7 |
| Cₘₐₓ (obs) | ng/g | 6828.8 | 1289.0 | 4037.2 | 1604.9 |
| Tₘₐₓ (obs) | hr | 1.0 | 4.0 | 1.0 | 2.0 |
| AUC(0-4) (obs area) | ng-hr/g | 12982.1 | 3004.1 | 7306.0 | 4184.4 |

Conclusions

In plasma, LL-oil showed a continuous release profile with constant increase of THC and CBD during the study period (240 min). In contrast, LL-P showed a biphasic release profile with immediate increase of THC and CBD during the first hour, followed by a decrease and another increase persisting until the study termination.

The PK profiles in the liver and brain reflected the plasma profiles. LL-P showed significantly more rapid absorption in tissues compared to LL-oil, for THC and CBD.

In the brain, Cmax values for CBD were significantly higher in LL-P compared to LL-oil (122.6 vs. 95.6 ng/g, respectively), the same was true for Cmax for THC in LL-P compared to LL-oil (206.9 vs. 115 ng/g, respectively). Similar phenomenon was observed in the liver.

These results suggest that with regard to oral bioavailability and tissues the LL-P composition is superior over LL-oil. Further, the LL-P composition was identified with a distinguishing bi-phasic release profile regarding both actives, THC and CBD, as opposed to a mono-phasic or continuous release profile of the LL-oil composition.

### Example 7: Bioavailability Studies with Vitamin D

The advantages of oral bioavailability of the compositions of the invention were further demonstrated in a study a rat model comparing the power compositions of the invention with vitamin D3 vs. conventional fat-soluble preparation.

Nanoemulsions were prepared as per standard protocol using both, lyophilization and spray drying. Table 14 shows the characteristic features of the obtained powder compositions.

TABLE 14
QC test of the powder composition with Vit. D3

| Vit. D powder | QC parameters | |
|---|---|---|
| Powder properties | Fine and white | |
| Vitamin D3 content % (w/w) | 300,000 | IU/g |
| Particle size-nm (in emulsion) | 150-200 | nm |

| | |
|---|---|
| Excipients | Disaccharide, Polysaccharide, |
| | Natural emulsifier |
| pH level in emulsion | 4.4 |
| Time to dissolution (sec) | <90 |
| Water content (%) | <2 |
| Flowability | Bulk density 0.5 gr/ml |
| | Tap density 0.7 gr/ml |
| | Angle of repose 45° |

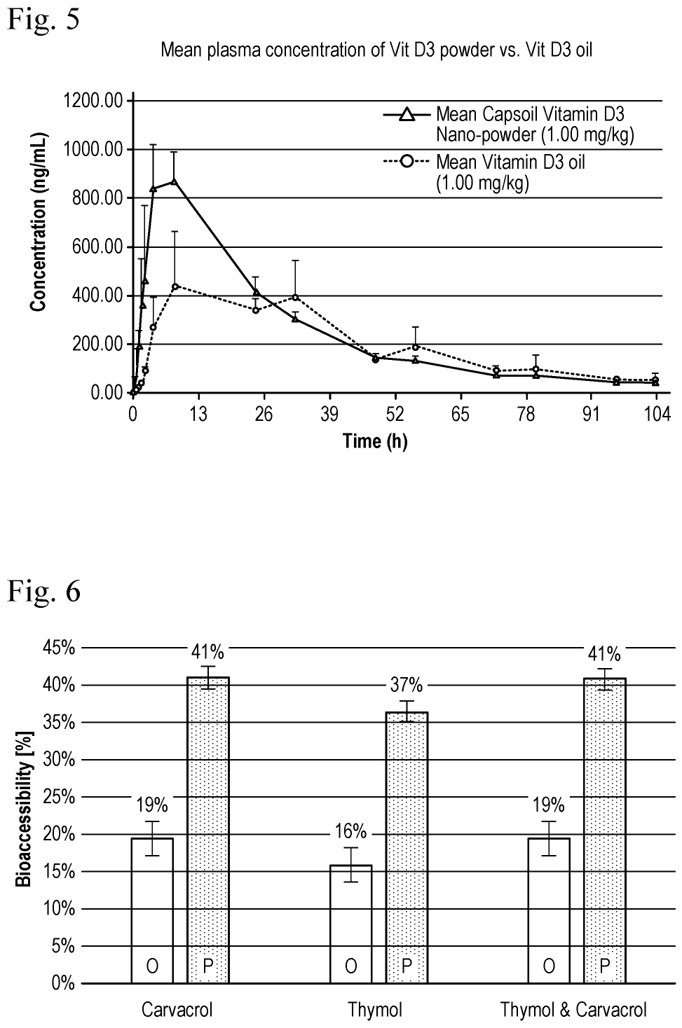

The pharmacokinetics assessment was performed in the rat plasma upon administration of a single oral dose of cholecalciferol (Vit D3) of 1 mg/kg body weight (N=9 per group). Blood samples were collected at Baseline=0 and 0.25 h, 0.5 h, 1 h, 1.5 h, 2 h, 4 h, 8 h, 24 h, 32 h, 48 h, 56 h, 72 h, 80 h, 96 h and 104 h (4 days). Steady-state cholecalciferol concentrations in plasma were measured by gas-liquid chromatography. Kinetic parameters were compared by both, after subtraction of Baseline concentrations and by using Baseline concentrations as a covariate. The results are shown in FIG. 5.

The results indicated that Vit D3 in the powder composition peaked rapidly reaching at a double concertation of the active in plasma relatively to the oil composition, and further remained at a steady state at a lower concertation for at least 60 h (3 days). The bioavailability of Vit D3 in the powder form as reflected in AUC (area under curve) was higher by 20%, and the half-life was longer by 15% (p<0.05) than in the oil form.

Overall, the results suggest improved bioavailability of lipophilic actives in the powder compositions of the invention with the features of an immediate and a prolonged release.

### Example 8: Enhanced Bio-Accessibility of Actives

### 8.1 Study In Vitro Mimicking the Conditions in the GI

The study explored the behavior of two actives, Thymol (2-isopropyl-5-methyl phenol) and Carvacrol (2-methyl5-(1-methylethyl) phenol), found in Oregano oil. Oregano oil is known for its beneficial properties, including antioxidant, free radical scavenging, anti-inflammatory, analgesic, antispasmodic, antibacterial, antifungal, antiseptic, and antitumor activities. Both these compounds have low solubility and permeability due to lipophilic properties and liability to degradation in the acidic condition in the stomach.

The study evaluated the bio-accessibility of Thymol and Carvacrol in the original oil form vs. the powder of the compositions of the invention using in vitro semi-dynamic digestion model. Bio-accessibility reflects the degree of GI digestion, i.e., an amount of compound released in the GI tract and becoming available for adsorption (e.g., enters the bloodstream). This parameter is further dependent on digestive transformation of the compound and its respective adsorption into intestinal cells and pre-systemic, intestinal, and hepatic metabolism. Bio-accessibility in vitro can be evaluated according to the following equation:

Bio-accessibility (%)=(Thymol and Carvacrol content after digestion in vitro/Thymol and Carvacrol initial content)×100

There are several types of in vitro digestion models: the static, semi-dynamic, and dynamic models. The static model is characterized by a single set of initial conditions (pH, concentration of enzymes, bile salts, etc.) for each part of the GI tract. It is relatively simplistic and has many advantages, but often provide a not realistic simulation of complex in vivo processes. The dynamic digestion model, in contrast, further includes corrections for geometry, biochemistry, and physical forces to better reflect in vivo digestion (e.g., continuous flow of the digestion content from the stomach to intestine, HCl addition, pepsin flow rate, gastric emptying, and controlled bile secretion). The semi-dynamic model is an intermediate model combining the advantages of both approaches. It includes pH modulation by HCl in the gastric phase and NH₄HCO₃ in the intestinal phase (unlike the static model) but has no continuous flow of the digestion contents and the intestinal stage begins after the gastric stage (unlike in the dynamic model).

Materials and Methods

Actives were tested in the forms of: (1) Oregano oil: 365 μl (˜300 mg Oregano oil) comprising 1.26 mg Thymol and 26.31 mg Carvacrol; and (2) Oregano powder: 1.11 gr the powder composition of the invention comprising 1.30 mg Thymol and 26.31 mg Carvacrol. The powder composition was produced according to the above method, yielding loading of 30% Oregano oil (w/w).

The two forms were tested in the semi-dynamic digestion system using INFOGEST protocol. The concentration of Thymol and Carvacrol was measured at the Baseline and after 2 h (representative of the end-gastric phase). Samples were analyzed by gas chromatography-mass spectrometry (GC-MS) using fused silica capillarity column (30 M, 0.25 mm), source temperature of 230° C., quad temperature of 150° C., and column oven temperature 250° C. for 3 min. Digesta sample (1 μl) was injected and concentration of analytes was calculated (peak area against standard peak area). The calibration curve showed linearity of the MS response. All preparations were analyzed by GC-MS before and after the in vitro gastric digestion at relevant time points. Chemical analysis of the oil and powder compositions was performed to assess loss of actives during powder preparation.

Results

Thymol and Carvacrol concentrations were reduced during the powder preparation process by 7% and 10%, respectively. In vitro digestions studies of the two forms showed that at the end of the gastric phase (2 h post-ingestion), the bio-accessibility of Carvacrol was 19% and 41% (more than twice) for the oil the powder forms, respectively. Similarly, the bio-accessibility of Thymol was 16% and 37% for the oil the powder forms. The bio-accessibility of both actives was 19% and 41% for the oil and powder forms, respectively. In other words, while only about 20% actives in the oil composition survived the acid pH in the stomach, the actives survival in the powder composition was significantly increased. The results are shown in FIG. 6.

Conclusions

Overall, the results suggest that the powder compositions of the invention can protect actives from gastric degradation, and thereby increase their oral bioavailability and bio-accessibility to the circulation and tissues.

### 6.2 Comparative Study Including Powders in Enteric-Coated Capsules

Analogous study was performed, including the oil and powder forms as above and the powder form in enteric-coated capsules (acid resistant coating). Thymol and Carvacrol concentrations were measured at Baseline and after 2 h (end of gastric phase), with calculations of bio-accessibility as above. In addition, the powder in enteric-coated capsules was shifted from the stomach phase to the duodenal phase and tested after 4 h (end of duodenal phase).

Results

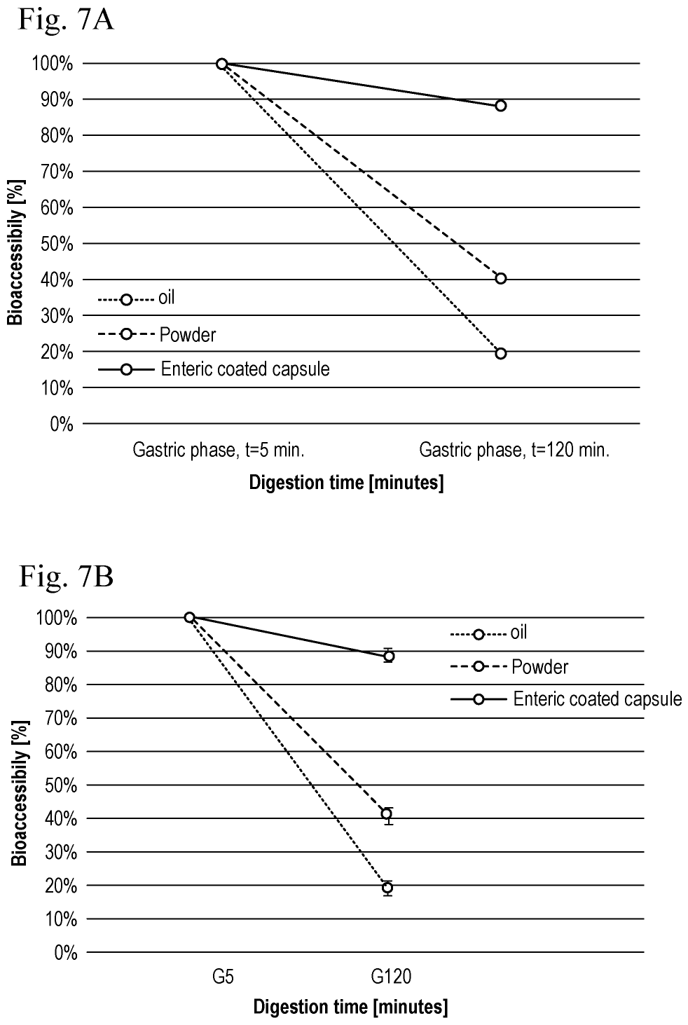

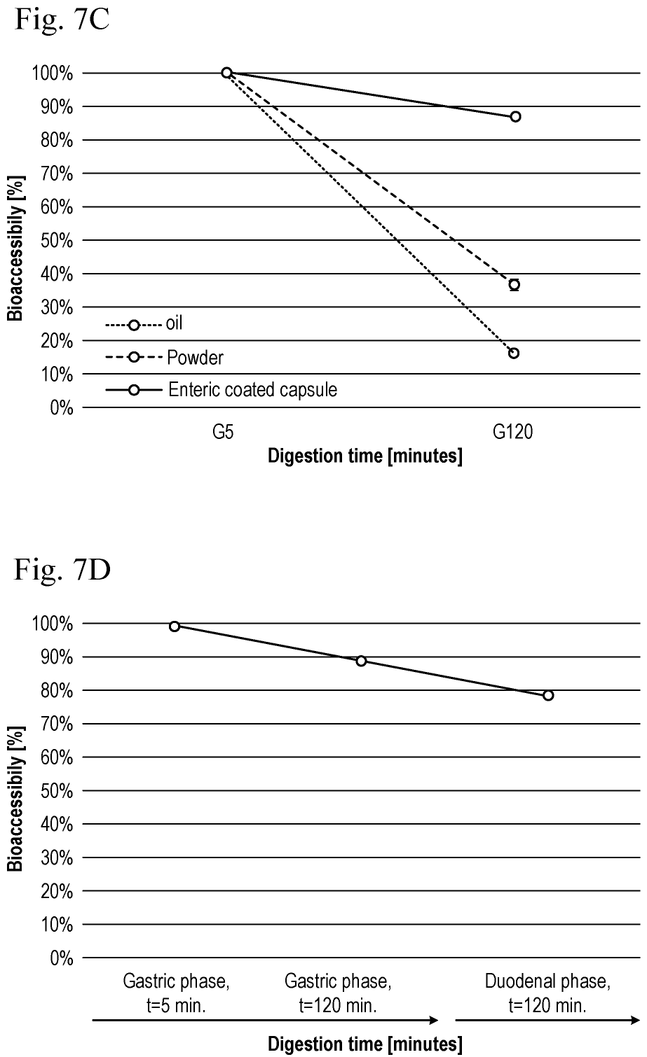

The bio-accessibility of Thymol and Carvacrol at the end of the gastric phase was 19%, 41% and 89% for the oil and powder forms and the powder in enteric coated capsules, respectively, suggesting significant differences between various types of compositions. Similar results were obtained for the separate actives. For Thymol for example, the bio-accessibility was 16%, 37% and 87%, respectively. The results are shown in FIGS. 7A-7C. The bio-accessibility of the powder in enteric coated capsules at the end of the duodenal phase was 79% (for both actives). The results are shown in FIG. 7D. The bio-accessibility of Carvacrol was 78% and Thymol 97%.

Conclusions

The results suggest that the protective effect of the powder compositions can be further enhanced by the addition of functional coating, thereby increasing even further their gastric and duodenal bio-accessibility.

Overall, the invention provides a highly relevant pharmaceuticals platform for formulating poorly water-soluble actives as oils to achieve improved oral bioavailability and bio-accessibility.

### Example 9: Compositions in Edible Products

In a preliminary trial, the powder CBD composition of the invention was used for the preparation of several food products: Pectin jelly, Nougat, Gums. Typical protocols are given below.

### 9.1 Pectin Jelly

1) Water (70 g) at 95° C.
2) Pectin solution: pectin (6 g)+4 g Tri-sodium citrate (4 g)+18 g sugar (18 g)
3) Citric acid 50% (5 g) containing the powder CBD composition (4 g)
4) Sugar (155 g) in a form of syrup
5) Sugar syrup was added to the pectin solution and cooked
6) Citric acid and CBD mix added with optional additives for color and flavor.

### 9.2 Nougat Formula

1) Solution 1: Water (287 g)+egg albumin (93 g)+syrup DE60 (320 g) mixed at 35° C. until homogenization
2) Solution 2: Water (397 g)+sugar (1600 g)+syrup (1150 g) cooked until evaporated (about 450 g)
3) Fat solution: A fat melted (60° C.)+milk powder+fondant powder+dried nut mix (200 g)
4) CBD solution (60%): the powder CBD composition (7.2 g) dispersed in water (5 g)
5) CBD solution added to Solutions 1 and 2
6) Fat solution is added to the mix.

### 9.3 Chewy Formula

1) Solution 1: Water+syrup+sorbitol mix (60° C.)
2) Solution 2: Sugar+starch+sisterna sp30 powder are added and mixed
3) Fat added, and the entire mixture is cooked (±120° C.)
4) CBD solution: Mixture of the powder CBD composition+color+acid+flavor
5) CBD solution added to Solutions 1 and 2
6) The product is cooled.

### Example 10. Micronized Sugar Particles of the Invention

### 10.1 Example Formulations

An example formulation of micronized sugar was produced, comprising sucrose, maltodextrin, sugar ester (SP30) and Theobroma oil. The amounts and the proportions of ingredients are detailed in Table 15. An example protocol of the process of making this type of formulation is listed further below.

TABLE 15
Amounts and concentrations of ingredients

| | | Concentration in the dry |
|---|---|---|
| Ingredient | Total amount (gr)* | formulation (% w/w) |
| Sucrose | 610 | 61 |
| Maltodextrin | 150 | 15 |
| Sugar ester (SP30) | 40 | 4 |
| Theobroma oil | 200 | 20 |
| Added water (DDW) | 2200 | NR |

*Total dry weight of all ingredients: 1000 gr

### Essential Steps in the Process of Making the Formulation Included:

i. Sucrose and maltodextrin were weighed and transferred to a container.
ii. DDW was added, the solution was stirred until the ingredients were dissolved.
iii. Sugar ester (Sp30) was weighed and added while stirring, the solution was heated to 50° C. for 5 min until the sugar ester was fully dissolved.
iv. Theobroma oil was weighed and added, the solution was stirred using Homogenizer to produce uniform emulsion.
v. The emulsion was fed to High Pressure Microfluidizer for 3 cycles (4 bar, pressure: 16,000 PSI) yielding nanodrops in the size range of about 100 nm-200 nm.
vi. The nanoemulsion was frozen (−30° C. or below) and placed in Lyophilizer until dried (about 2 days at 0.04 mBar or below). Alternatively, the frozen nanoemulsion was spray dried at about 190° C.

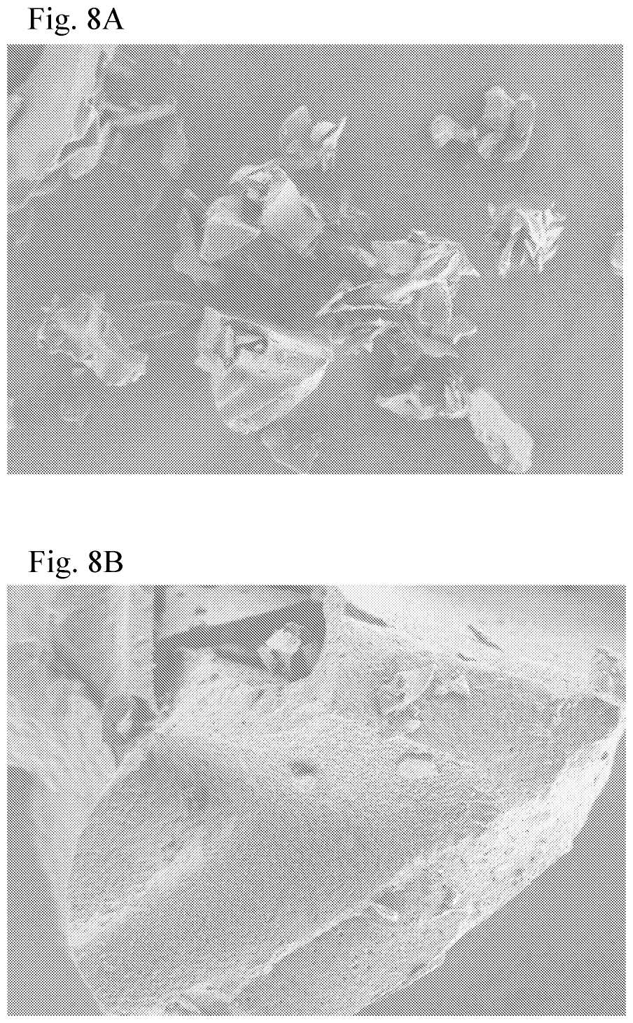

The powder product was analyzed by Scanned Electron Microscope (SEM). SEM images in FIGS. 8A-8B show a smooth finely granulated sugar particles with size in the range of 20-50 μm. Overall, the results show that the sugar powder the invention was relatively uniform in terms of texture and size, with smooth and finely granulated particles below 50 μm.

### 10.2 Entrapment of Nanometric Oil Drops in the Sugar Particle

Morphological characterization of the sugar particles with vitamin E oil was performed Cryogenic Transmission Electron Microscopy (cryo-TEM). Samples were prepared in Controlled Environment Vitrification System (CEVS) with humidity at saturation to prevent evaporation of volatiles and temperature of 25° C.

The solution (1 drop) was placed on carbon-coated perforated polymer film supported on 200 mesh TEM grid. The drop was converted to a thin film (<300 nm) by removing excess solution. The grid cooled in liquid ethane at −183° C. Cryo-TEM imaging was performed on Thermo-Fisher Talos F200C at 200 kV. Micrographs were recorded by Thermo-Fisher Falcon III direct detector camera (4 k×4 k resolution). Samples were examined in TEM nanoprobe mode using volta phase plates. Imaging was performed at low dose mode and acquired by TEM TIA software.

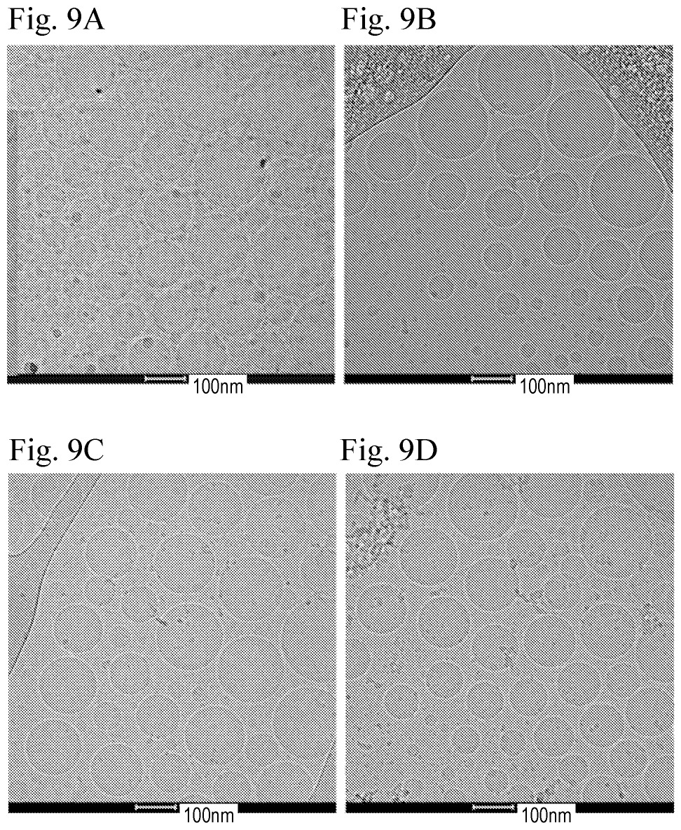

Images of the cryo-TEM sections in FIGS. 9A-9D show bright and smooth surfaced spherical nano-droplets with size in the range of 80-150 nm. Overall, the results indicate entrapment of spherical nanometric oil drops in the particle, the oil drops had a relatively uniform size below 150 nm.

### 10.3 Controlling the Sugar Particle Size by the Size Lipophilic Nanodrops

Co-relationship between the sugar particle size and the size of lipophilic nanodrops was demonstrated in the example of the Theobroma oil formulation. The size of lipophilic nanodrops was modified by variation of cycles and/or intensity in the homogenization step (see 10.1).

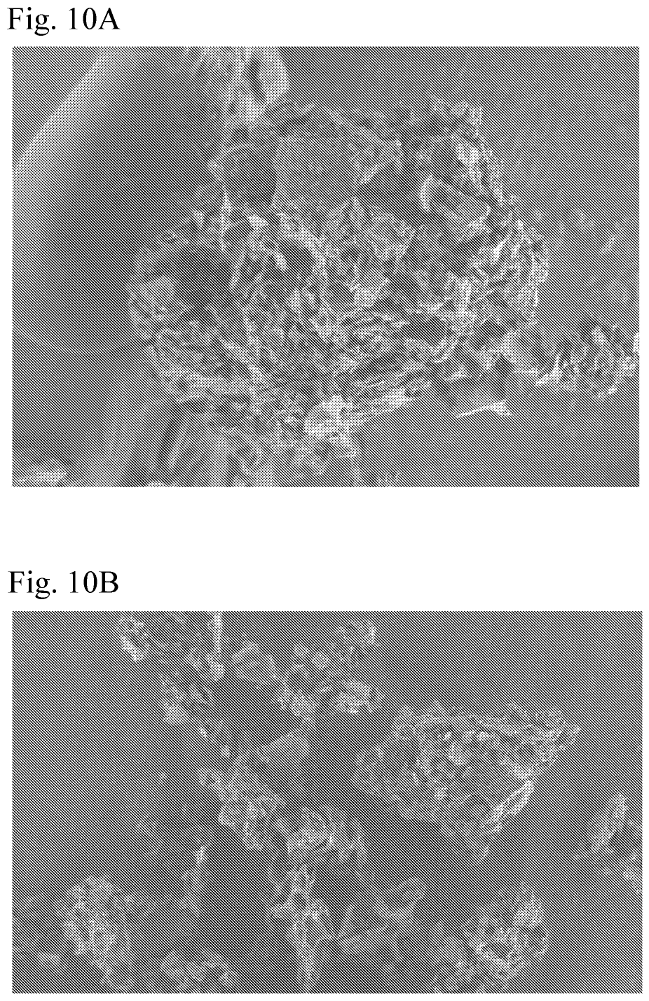

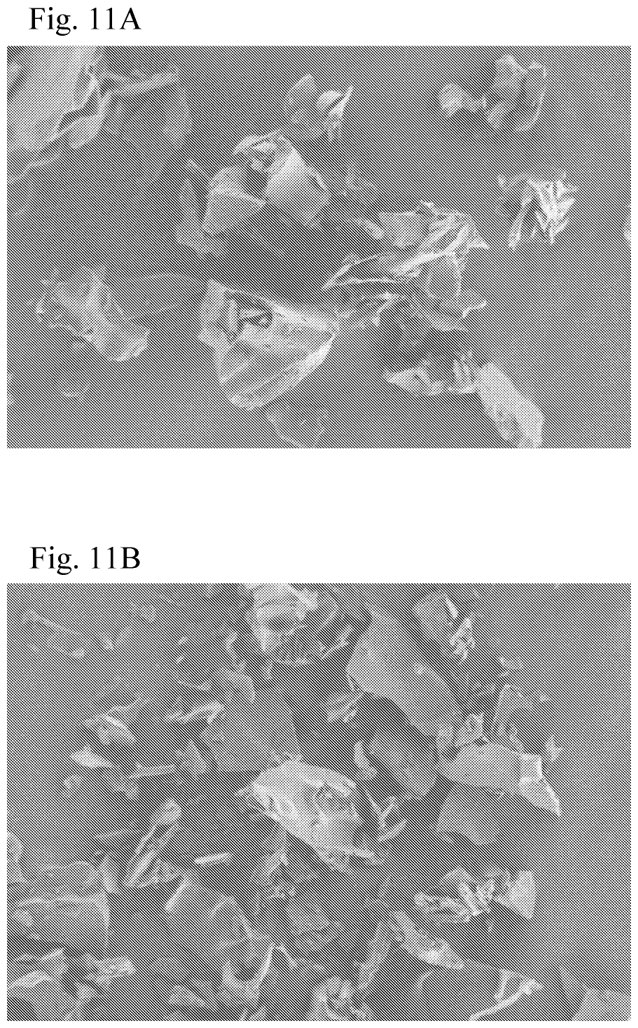

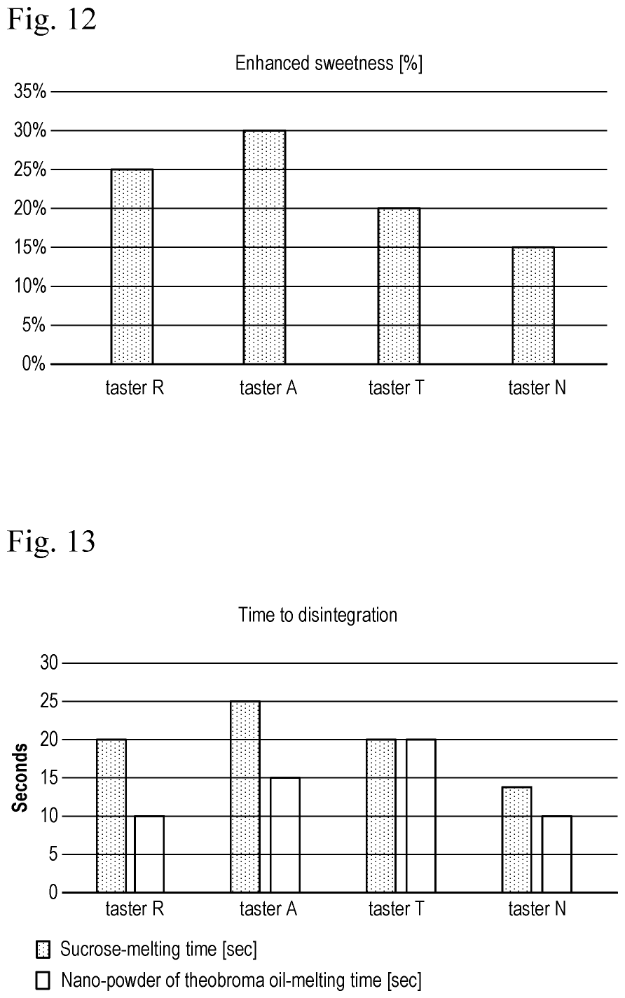

The powder products were analyzed by SEM. SEM images in FIGS. 10A-10B and 11A-11B show sugar particles produces under various emulsification conditions. While the lipophilic nanodrops with an average size of about 800 nm yielded sugar particles with size in the range of 130-160 μm, the lipophilic nanodrops with an average size of about 150 nm yielded sugar particles with size in the range of 20-50 μm.

This experiment has provided evidence that the size of the entrapped nanodrops impacts the size of the sugar particles. The nano-emulsions with larger nanodrops produce larger sugar particles and finer nano-emulsions produce finer sugar particles, with specific examples of particles with size in the range of about 130 μm to 160 μm and in the range of about 20 μm to 50 μm. The overall conclusion is that the size of the sugar particles can be modulated by modulating the size of the entrapped nanodrops.

10.4 Organoleptic Properties of the Formulation with Theobroma Oil

Advantageous features of the formulation with Theobroma oil were demonstrated in an organoleptic test by 4 tasters, comparing the sensation of sweetness and melting in the mouth with the formulation of the invention vs. sucrose. The results are shown in Tables 16 and 17 below and in FIGS. 12 and 13.

TABLE 16
Comparative organoleptic test of sweetness

| | Enhances sweetness in |
|---|---|
| | Theobroma oil formulation (%) |
| Taster R | 25 |
| Taster A | 30 |
| Taster T | 20 |
| Taster N | 15 |

TABLE 17
Comparative organoleptic test of melting sensation

Melting time (sec)

| | | Theobroma oil |
|---|---|---|
| | Sucrose | formulation |
| Taster R | 20 | 10 |
| Taster A | 25 | 15 |
| Taster T | 20 | 20 |
| Taster N | 14 | 10 |

The comparative test of the sensation of sweetness showed that by all tasters the formulation of the invention had an enhanced sweetness up to at least 15% to 30%. More than sucrose. The sensation of melting or disintegration in the mouth showed that by all taster the melting time of the formulation of the invention was faster than sucrose.

Overall, the results suggest that the formulation of Theobroma oil of the invention, due to its particular structure and morphology, demonstrates superior features of enhanced sweetness and sensation of meting in the mouth compared to regular sugar. These two features, in particular, are considered an advantageous combination for many types of desserts and fondants, and especially for various types of chocolates.

10.5 Dissolution Analysis of the Formulation with Theobroma Oil

The feature of enhanced disintegration was further demonstrated in an objective test comparing the dissolution rate of 4 types of powders:
(A) Powder of Sucrose: Maltodextrine (8:2 w/w)
(B) Powder of finely crushed Sucrose: Maltodextrine (8:2 w/w)
(C) Micropowder with Theobroma oil
(D) Nanopowder with Theobroma oil

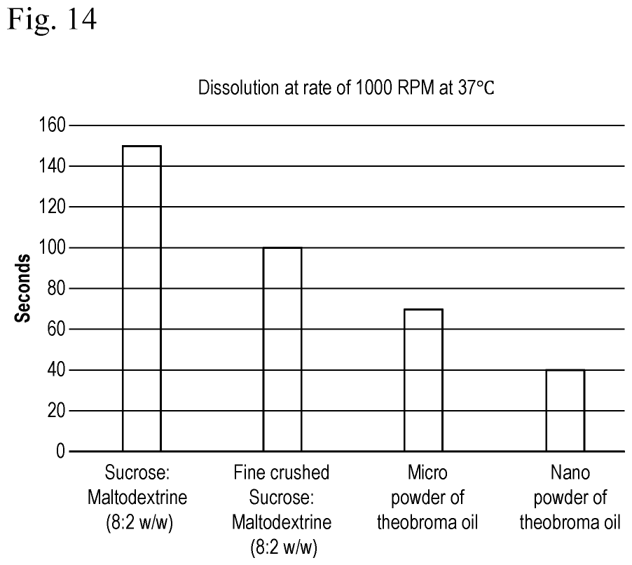

The dissolution test was performed under stirring at the rate of 1000 RPM and 37° C. The results are shown in Table 18 and FIG. 14.

TABLE 18
Comparative dissolution test of various powders

| | Powder A | Powder B | Powder C | Powder D |
|---|---|---|---|---|
| Time (sec) | 150 | 100 | 70 | 40 |

The comparative dissolution test showed that the nanopowder formulation of the invention with Theobroma oil had significantly faster disintegration time compared to the other types of tested powders, providing a further reinforcement of the previous organoleptic test.

### Annex A

Major Edible Oils
Coconut oil, an oil high in saturated fat
Corn oil, an oil with little odor or taste
Cottonseed oil, an oil low in trans-fats
Canola oil, (a variety of rapeseed oil)
Olive oil
Palm oil, the most widely produced tropical oil
Peanut oil (ground nut oil)
Safflower oil
Sesame oil, including cold pressed light oil and hot pressed darker oil
Soybean oil, produced as a byproduct of processing soy meal
Sunflower oil

Edible Nut Oils
Almond oil
Cashew oil,
Hazelnut oil
Macadamia oil, has no trans-fats, and a good balance omega-3/omega-6
Pecan oil
Pistachio oil
Walnut oil

Nutrient Rich Oils
Amaranth oil, high in squalene and unsaturated fatty acids
Apricot oil
Argan oil, a food oil from Morocco
Artichoke oil, extracted from the seeds of Cynara cardunculus
Avocado oil
Babassu oil, a substitute for coconut oil
Ben oil, extracted from the seeds of Moringa oleifera
Borneo tallow nut oil, extracted from the fruit of Shorea
Buffalo gourd oil, extracted from the seeds of Cucurbita foetidissima
Carob pod oil (Algaroba oil)
Coriander seed oil
False flax oil made of the seeds of Camelina sativa
Grape seed oil
Hemp oil, a high quality food oil
Kapok seed oil
Lallemantia oil, extracted from the seeds of Lallemantia iberica
Meadowfoam seed oil, highly stable with over 98% long-chain fatty acids
Mustard oil (pressed)
Okra seed oil, extracted from the seed of Hibiscus esculentus
Perilla seed oil, high in omega-3 fatty acids
Pequi oil, extracted from the seeds of Caryocar brasiliensis
Pine nut oil, an expensive food oil from pine nuts
Poppyseed oil
Prune kernel oil, a gourmet cooking oil.
Pumpkin seed oil, a specialty cooking oil
Quinoa oil, similar to corn oil
Ramtil oil, pressed from the seeds of Guizotia abyssinica (Niger pea)
Rice bran oil
Tea oil (Camellia oil)
Thistle oil, pressed from the seeds of Silybum marianum.

Natural Edible Sugars
Beet sugar, white and granulated sugar
Cane sugar, white refined or brown sugar
Brown sugar, granulated cane sugar that has molasses (dark and light brown)
Demerara sugar, a type of raw cane sugar
Fructose, fruit sugar twice as sweet as refined cane sugar
Fruit sweetener (liquid and solid) made from grape juice concentrate blended with rice syrup
Jaggery (palm sugar, gur), made from the reduced sap of either the sugar palm or the palmyra palm
Maple sugar, much sweeter than white sugar and has fewer calories
Muscovado (Barbados) sugar, a raw cane sugar similar to brown sugar
Piloncillo (panela, panocha), another type of a raw cane sugar
Rock sugar (Chinese rock sugar), a lightly caramelized cane sugar
Sucanat: juice from organically grown sugarcane turned into granular sugar
Turbinado sugar, raw cane sugar crystals derived from sugarcane
White refined sugar (granulated sugar, table sugar, sucrose) derived from sugarcane or sugar beets

Natural Liquid Sweeteners
Barley malt syrup
Corn syrup
Honey
Malt syrup (malt extract)
Maple syrup (Grades A, B and C)
Maple honey
Molasses
Rice syrup
Sorghum molasses (sorghum syrup

Sugar Substitutes
Advantame, artificial sweetener approved by the FDA
Acesulfame-K, artificial sweetener approved by the FDA
Agave syrup, taken from the nectar of the agave cactus
Aspartame, artificial sweetener approved by the FDA, contains amino acids
Neotame, artificial sweetener approved by the FDA
Saccharine, artificial sweetener
Sorbitol, occurs naturally in some fruits and berries.
Stevia, an herbal extract from a member of the chrysanthemum family,
Sucralose, a chemically modified sugar approved by the FDA.

Edible Polysaccharides
Starch, generally a polymer consisting of two amylose (normally 20-30%) and amylopectin (normally 70-80%) primarily found in cereal grains and tubers like corn (maize), wheat, potato, tapioca, and rice
Kaempferia rotunda and Curcuma xanthorrhiza essential oils that are enriched in cassava starch-based polysaccharide
Maltodextrin, a polysaccharide produced from vegetable starch
Alginate, a naturally occurring anionic polymer obtained from brown seaweed, also used in various pharmaceutical preparations such as gaviscon, bisodol, and asilone
Carrageenans, water-soluble polymers with a linear chain of partially sulfated galactans
Pectins, a group of plant-derived polysaccharides
Agars, hydrophilic colloids that have the ability to form reversible gels
Chitosan, a promising group of natural polymers with characteristics such as biodegradability, chemical inertness, biocompatibility, high mechanical strength
Gums, edible-polymer preparations used for their texturizing capabilities
Certain cellulose derivative forms, predominantly four are used in the food industry: hydroxypropyl cellulose (HPC), hydroxypropyl methylcellulose (HPMC), carboxymethylcellulose (CMC), or methylcellulose (MC).

Food Emulsifiers
lecithin and lecithin derivatives
glycerol fatty acid esters
hydroxycarboxylic acid and fatty acid esters
lactylate fatty acid esters
polyglycerol fatty acid esters
ethylene or propylene glycol fatty acid esters
ethoxylated derivatives of monoglycerides

Natural and Nature-Identical Colorants Allowed in the EU and the USA
Curcumin (Turmeric
Riboflavin
Cochineal, Cochineal extract, carminic acid, carmines
Chlorophyll(in)s copper complexes chlorophyll(in)s
Caramel
Vegetable carbon
Carrot oil, β-carotene
Annatto, bixin, norbixin
Paprika extract
Lycopene
β-Apo-8′-carotenal
Ethyl ester of β-apo-8′-carotenoic acid
Lutein
Canthaxanthin
Beetroot red
Anthocyanins
Cottonseed flour
Vegetable juice
Saffron

Acidulants and Other Preservatives
Lactic acid, acetic acid and other acidulants, alone or in conjunction with other preservatives such as sorbate and benzoate
Malic and tartaric (tartric) acids
Citric acid
Ascorbic acid/vitamin C, isoascorbic isomer, erythorbic acid and their salts

Lipophilic Food Preservatives
Benzoic acid in the form of its sodium salt
Sorbic acid and potassium sorbate, specifically for mold and yeast inhibition
Lipophilic arginine esters, a more recent group of compounds

1. An oral solid water-dispersible powder composition of matter comprising at least one sugar, at least one polysaccharide and at least one surfactant and at least one edible lipophilic substance which is at least one edible oil or is dissolved in at least one edible oil,
wherein the composition comprises a plurality of micrometric particles each comprising a plurality of lipophilic nanospheres with an average size in the range of about 50 nm to about 900 nm, the at least one edible lipophilic substance is contained in the micrometric particles and is distributed inside and/or outside the lipophilic nanospheres at predetermined proportions,
wherein the composition has an encapsulation capacity of the at least one edible lipophilic substance of 70 to 98% (w/w) relative to the weight of the oil component, and
wherein the size of the nanospheres is substantially maintained upon dispersion of the powder composition in water,
thereby providing improved delivery of the at least one edible lipophilic substance.

2-5. (canceled)

6. The composition of claim 1 having a loading capacity of the at least one edible lipophilic substance up to about 50% (w/w) relative to total weight.

7. (canceled)

8. (canceled)

9. The composition of claim 1, wherein the micrometric particles have an average size between about 10 μm and to about 300 μm.

10. (canceled)

11. (canceled)

12-13. (canceled)

14. The composition of claim 1, wherein the at least one edible oil is a natural oil obtained from a vegetable or an animal source, or a synthetic oil or a fat, which are solid, semi-solid or liquid at room temperature, or a mixture thereof.

15. (canceled)

16. The composition of claim 14, wherein the at least one edible oil is selected from canola oil, sunflower oil, sesame oil, peanut oil, grapeseed oil, ghee, avocado oil, coconut oil, pumpkin seed oil, flaxseed oil, hemp oil, and olive oil.

17. (canceled)

18. The composition of claim 1, wherein the at least one edible polysaccharide is selected from maltodextrin and carboxymethyl cellulose (CMC).

19. (canceled)

20. The composition of claim 1, wherein the at least one edible surfactant is selected from a monoglyceride, a diglycerine, a glycolipid, a lecithin, a fatty alcohol, a fatty acid, and a sucrose fatty acid ester (sugar ester), or a mixture thereof.

21. (canceled)

22. The composition of claim 1, wherein the at least one edible lipophilic substance constitutes between about 10% to about 98% of the composition (w/w).

23. (canceled)

24. The composition of claim 1, wherein the at least one edible lipophilic substance is selected from beneficial oils, nutraceuticals, vitamins, dietary or food supplements, nutrients, antioxidants, superfoods, natural extracts of animal or plant origin, and probiotic microorganisms, or a combination thereof.

25. (canceled)

26. The composition of claim 1, wherein the improved delivery of the at least one edible lipophilic substance comprises at least one of:
(i) an improved oral bioavailability of the at least one edible lipophilic substance in plasma or at least one tissue;
(ii) an improved bio-accessibility of the at least one edible lipophilic substance into at least a part of the gastrointestinal (GI) tract or at least one tissue in the GI tract;
(iii) an improved permeation of the at least one edible lipophilic substance into at least a part of the gastrointestinal (GI) tract or at least one tissue; and
wherein the composition is adapted for oral, sublingual, or buccal administration.

27-38. (canceled)

39. A food, a beverage, a food supplement, a candy product, a lozenge, or a bubble gum comprising the composition of claim 1 or a dispersion of the composition of claim 1.

40-77. (canceled)

78. The composition of claim 1, wherein the at least one edible sugar is selected from oligo-, di-, monosaccharides and polyols, or a mixture thereof.

79. The composition of claim 1, wherein the at least one edible sugar is selected from trehalose, sucrose, glucose, fructose, maltose, galactose, mannitol, lactitol and lactose, or a mixture thereof.

80. The composition of claim 1, wherein the at least one edible surfactant is a food emulsifier.

81. The composition of claim 80, wherein the food emulsifier is selected from ammonium glycyrrhizinate, pluronic F-127 and pluronic F-68.

82. The composition of claim 1, wherein the at least one edible sugar constitutes between about 10% to about 90% of the composition (w/w).

83. The composition of claim 1, having a long-term stability of at least about 1 year at room temperature.

84. The composition of claim 1, wherein the size of the nanospheres is substantially maintained upon lyophilization, long-term storage, fixation and release from matrixes or films.

85. A dispersion comprising the composition of claim 1.

86. A food additive, a food colorant, a taste or aroma enhancer, a taste masker, a food preservative, or a composition thereof, comprising the composition of claim 1 or a dispersion of the composition of claim 1.
